(19) United States
(12) Patent Application Publication
(10) Pub. No.: US 2011/0190274 A1
(43) Pub. Date: Aug. 4, 2011

(54) SALT OF, AND PROCESSES FOR THE PREPARATION OF, 1-ISOPROPYL-4-HEXAHYDRO-1H-1,4-DIAZEPINE

(75) Inventors: Caroline Jane Day, Hertfordshire (GB); Trevor Raymond Keel, Hertfordshire (GB); Geracimos Rassias, Hertfordshire (GB)

(73) Assignee: GLAXO GROUP LIMITED

(21) Appl. No.: 13/058,559

(22) PCT Filed: Aug. 14, 2009
(86) PCT No.: PCT/EP2009/060578

§ 371 Date: Apr. 6, 2011

(30) Foreign Application Priority Data
Aug. 15, 2008 (GB) 0814997.3
Mar. 12, 2009 (GB) 0904311.8

(51) Int. Cl. A61K 31/551; C07D 243/08; A61P 11/00

(52) U.S. Cl. 514/218; 540/575

(57) ABSTRACT

The invention relates to 1-isopropyl-4-{[4-(tetrahydro-2H-pyran-4-yloxy)phenyl]carbonyl}hexahydro-1H-1,4-diazepine mono-maleate, and crystalline Form 1 thereof.

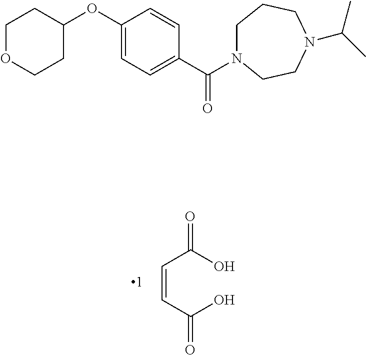

The present invention relates to a salt of, and processes for the preparation of, a hexahydro-1H-1,4-diazepine derivative having affinity for the histamine H3 receptor (which is 1-isopropyl-4-{[4-(tetrahydro-2H-pyran-4-yloxy)phenyl]carbonyl}hexahydro-1H-1,4-diazepine); to processes for the preparation of an intermediate(s) useable in the preparation of the hexahydro-1H-1,4-diazepine derivative; and to an intermediate useable in the preparation of the hexahydro-1H-1,4-diazepine derivative.

### BACKGROUND OF THE INVENTION

The histamine H3 receptor is predominantly expressed in the mammalian central nervous system (CNS), with minimal expression in peripheral tissues except on some sympathetic nerves (Leurs et al., (1998), Trends Pharmacol. Sci. 19, 177-183). Activation of H3 receptors by selective agonists or histamine results in the inhibition of neurotransmitter release from a variety of different nerve populations, including histaminergic and cholinergic neurons (Schlicker et al., (1994), Fundam. Clin. Pharmacol. 8, 128-137). Additionally, in vitro and in vivo studies have shown that H3 antagonists can facilitate neurotransmitter release in brain areas such as the cerebral cortex and hippocampus, relevant to cognition (Onodera et al., (1998), In: The Histamine H3 receptor, ed. Leurs and Timmerman, pp 255-267, Elsevier Science B.V.). Moreover, a number of reports in the literature have demonstrated the cognitive enhancing properties of H3 antagonists (e.g. thioperamide, clobenpropit, ciproxifan and GT-2331) in rodent models including the five choice task, object recognition, elevated plus maze, acquisition of novel task and passive avoidance (Giovanni et al., (1999), Behav. Brain Res. 104, 147-155). The histamine H3 receptor antagonist GSK189254 inhibited [3N]R-α-methylhistamine ex vivo binding in the rat cortex following oral administration to the rat, and at certain oral doses improved performance of rats in the following cognition paradigms: passive avoidance, water maze, object recognition, and attentional set shift (A. D. Medhurst et al., J. Pharmacol. Exp. Therap., 2007, 321(3), 1032-1045.).

WO 2005/040144 A1 (Glaxo Group Limited) discloses a series of 1-benzoyl-substituted diazepanyl derivatives or a pharmaceutically acceptable salt thereof having affinity for and being antagonists and/or inverse agonists of the histamine H3 receptor, and which are stated therein to be believed to be of potential use in the treatment of neurological diseases including Alzheimer's disease, dementia (including Lewy body dementia and vascular dementia), age-related memory dysfunction, mild cognitive impairment, cognitive deficit, epilepsy, neuropathic pain, inflammatory pain, migraine, Parkinson's disease, multiple sclerosis, stroke and sleep disorders (including narcolepsy and sleep deficits associated with Parkinson's disease); psychiatric disorders including schizophrenia (particularly cognitive deficit of schizophrenia), attention deficit hyperactivity disorder, depression, anxiety and addiction; and other diseases including obesity and gastro-intestinal disorders. WO 2005/040144 A1 discloses this series of compounds or salts thereof for use as a therapeutic substance in the treatment or prophylaxis of the above disorders, in particular cognitive impairments in diseases such as Alzheimer's disease or a related neurodegenerative disorder.

Example 10 of WO 2005/040144 A1 discloses the preparation of 1-(isopropyl)-4-{[4-(tetrahydro-2H-pyran-4-yloxy)phenyl]carbonyl}hexahydro-1H-1,4-diazepine hydrochloride:

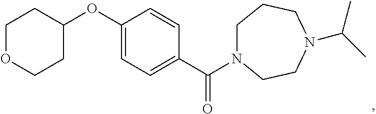

using the following method:

A stirred suspension of 4-(tetrahydro-2H-pyran-4-yloxy)benzoic acid (D6) (222 mg) in dichloromethane (5 ml) at room temperature was treated with oxalyl chloride (0.28 ml) and 10% dimethylformamide in dichloromethane (1 drop). After 1 h the solution was evaporated and then re-evaporated from dichloromethane (2×5 ml). The acid chloride was redissolved in dichloromethane (10 ml) and treated with 1-(isopropyl)-hexahydro-1H-1,4-diazepine dihydrochloride . . . (178 mg) and diethylaminomethyl polystyrene (3.2 mmol/g, 938 mg). After stirring overnight the mixture was loaded directly on to a silica gel flash column [step gradient 6-10% MeOH (containing 10% 0.880 ammonia solution) in dichloromethane]. Fractions containing the required product were evaporated, then redissolved in dichloromethane and treated with excess 4M HCl in dioxan. Crystallisation from acetone afforded the title compound (E10) (225 mg). MS electrospray (+ion) 347 (MH⁺). ¹H NMR δ (DMSO-d6): 10.45 (1H, m), 7.41 (2H, d, J=8.5 Hz), 7.02 (2H, d, J=8.5 Hz), 4.63 (2H, m), 4.02 (1H, m), 3.02-3.93 (13H, m), 2.32 (1H, m), 1.96 (2H, m), 1.61 (2H, m), 1.27 (6H, d, J=6.5 Hz).

WO 2005/040144 A1 discloses the preparation of the intermediate 4-(tetrahydro-2H-pyran-4-yloxy)benzoic acid (D6) as follows:

A solution of ethyl 4-(tetrahydro-2H-pyran-4-yloxy)benzoate (D5) (0.73 g) in ethanol (10 ml) was treated with 1M NaOH (5.84 ml) and the mixture stirred at 60° C. for 5 h. The solution was cooled to room temperature and the ethanol was evaporated. The aqueous was washed with dichloromethane (2×10 ml) and acidified. The solid was filtered off, washed with water and dried to give the title compound (D6) (0.55 g). MS electrospray (−ion) 221 (M-H). ¹H NMR δ (DMSO-d6): 7.87 (2H, d, J=8.5 Hz), 7.05 (2H, d, J=8.5 Hz), 4.69 (1H, m), 3.85 (2H, m), 3.50 (2H, m), 1.98 (2H, m), 1.59 (2H, m).

WO 2005/040144 A1 discloses the preparation of the intermediate ethyl 4-(tetrahydro-2H-pyran-4-yloxy)benzoate (D5) as follows:

An ice-cold solution of ethyl 4-hydroxybenzoate (0.82 g), 4-hydroxy-tetrahydro-2H-pyran (0.5 g) and triphenylphosphine in tetrahydrofuran (50 ml) was treated dropwise with diisopropyl azodicarboxylate (1.69 ml). After 15 min the cooling bath was removed and the reaction stood overnight at room temperature. The mixture was evaporated, redissolved in toluene and successively washed with 2N sodium hydroxide (2×20 ml), water (2×20 ml) and brine (20 ml). After drying (magnesium sulfate) the solution was loaded directly on to a silica flash column (step gradient 10-30% ethyl acetate in light petroleum 40-60) to give the title compound (D5) (0.75 g). ¹H NMR δ (CDCl₃): 7.98 (2H, d, J=8.5 Hz), 6.91 (2H, d, J=8.5 Hz), 4.60 (1H, m), 4.35 (2H, q, J=9.8 Hz), 3.98 (2H, m), 3.57 (2H, m), 2.05 (2H, m), 1.80 (2H, m), 1.38 (3H, t, J=9.8 Hz).

Copending PCT application PCT/EP2008/061664 (Glaxo Group Limited), filed on 4 Sep. 2008 and published as WO 2009/030716 A1 on 12 Mar. 2009, discloses 1-(1-methylethyl)-4-{[4-(tetrahydro-2H-pyran-4-yloxy)phenyl]carbonyl}piperazine

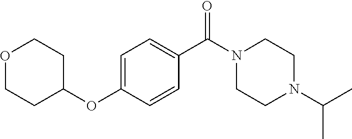

or a salt thereof, it use as an antagonist and/or inverse agonist of the histamine H3 receptor, and processes for the preparation of the compound or salt.

### BRIEF SUMMARY OF THE INVENTION

We have now discovered, inter alia, a new salt of 1-isopropyl-4-{[4-(tetrahydro-2H-pyran-4-yloxy)phenyl]carbonyl}hexahydro-1H-1,4-diazepine and a crystalline form thereof.

Therefore, a first aspect of the present invention provides 1-isopropyl-4-{[4-(tetrahydro-2H-pyran-4-yloxy)phenyl]carbonyl}hexahydro-1H-1,4-diazepine mono-maleate,

In a second aspect, the present invention provides crystalline Form 1 of 1-isopropyl-4-{[4-(tetrahydro-2H-pyran-4-yloxy)phenyl]carbonyl}hexahydro-1H-1,4-diazepine mono-maleate.

Crystalline Form 1 of 1-isopropyl-4-{[4-(tetrahydro-2H-pyran-4-yloxy)phenyl]carbonyl}hexahydro-1H-1,4-diazepine mono-maleate can be characterized by its spectroscopic and/or physical characteristics, such as by its X-ray powder diffraction (XRPD) diffractogram (defined in the third and fourth aspects of the invention hereinbelow), by its solid-form infrared (IR) spectrum (defined in the fifth and sixth aspects of the invention hereinbelow), and/or by its differential scanning calorimetry (DSC) thermogram (defined in the seventh aspect of the invention hereinbelow).

Other aspects of the invention provide, inter alia, processes for the preparation of 1-isopropyl-4-{[4-(tetrahydro-2H-pyran-4-yloxy)phenyl]carbonyl}hexahydro-1H-1,4-diazepine

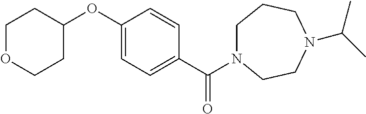

or a pharmaceutically acceptable salt thereof (such as the mono-maleate or hydrochloride salt), and processes for the preparation of chemical intermediates useful in the preparation of 1-isopropyl-4-{[4-(tetrahydro-2H-pyran-4-yloxy)phenyl]carbonyl}hexahydro-1H-1,4-diazepine, as defined in more detail hereinbelow.

Another aspect of the invention provides 1-isopropyl-hexahydro-1H-1,4-diazepine bis-trifluoroacetate, having the following formula, for example as a chemical intermediate:

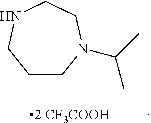

### BRIEF DESCRIPTION OF THE DRAWINGS

FIG. 1 is an X-ray powder diffraction (XRPD) diffractogram of the crystalline Form 1 of 1-isopropyl-4-{[4-(tetrahydro-2H-pyran-4-yloxy)phenyl]carbonyl}hexahydro-1H-1,4-diazepine mono-maleate prepared in Example 4, showing the two-theta angles (in degrees) on the horizontal axis plotted against the intensity (in counts) on the vertical axis, and obtained with a diffractometer using copper K-alpha X-radiation, with a step size of 0.0167° two-theta, a time per step of 31.75 sec, and using a sample mounted on a silicon wafer (zero background) plate (see Example 5.1 for details).

FIG. 2 is a solid-form attenuated total reflectance (ATR) infrared (IR) spectrum of the crystalline Form 1 of 1-isopropyl-4-{[4-(tetrahydro-2H-pyran-4-yloxy)phenyl]carbonyl}hexahydro-1H-1,4-diazepine mono-maleate prepared in Example 3, showing the spectral region from 4000 to 650 cm⁻¹ (see Example 3 for details).

FIG. 3 is a solid-form attenuated total reflectance (ATR) infrared (IR) spectrum of the crystalline Form 1 of 1-isopropyl-4-{[4-(tetrahydro-2H-pyran-4-yloxy)phenyl]carbonyl}hexahydro-1H-1,4-diazepine mono-maleate prepared in Example 3, showing the “fingerprint” spectral region from 1800 to 650 cm⁻¹ (see Example 3 for details).

FIG. 4 is a differential scanning calorimetry (DSC) thermogram of the crystalline 1-isopropyl-4-{[4-(tetrahydro-2H-pyran-4-yloxy)phenyl]carbonyl}hexahydro-1H-1,4-diazepine mono-maleate prepared in Example 4, measured at atmospheric pressure and under a flow of nitrogen using a calorimeter using a heating rate of about 10° C. per minute (see Example 5.2 for details).

FIG. 5 is a gravimetric vapour sorption (GVS) graph of the crystalline Form 1 of 1-isopropyl-4-{[4-(tetrahydro-2H-pyran-4-yloxy)phenyl]carbonyl}hexahydro-1H-1,4-diazepine mono-maleate prepared in Example 4, plotting the mass relative to the minimum mass (%) on the vertical axis versus relative humidity on the horizontal axis, measured at 25° C. under nitrogen (see Example 5.3 for details).

FIG. 6 is a gravimetric vapour sorption (GVS) graph of the “kinetic solid” 1-isopropyl-4-{[4-(tetrahydro-2H-pyran-4-yloxy)phenyl]carbonyl}hexahydro-1H-1,4-diazepine hydrochloride prepared in Example 2 Part C (the sample before purification by recrystallisation), plotting the mass relative to the minimum mass (%) on the vertical axis versus relative humidity on the horizontal axis, measured at 25° C. under nitrogen (see Example 5.4 for details).

FIG. 7. is a ¹H nuclear magnetic resonance (NMR) spectrum in d6-DMSO solution of 1-isopropyl-4-{[4-(tetrahydro-2H-pyran-4-yloxy)phenyl]carbonyl}hexahydro-1H-1,4-diazepine mono-maleate prepared in Example 4, showing chemical shifts in parts per million (ppm) plotted versus normalised intensity (see Example 5.5 for details).

### DETAILED DESCRIPTION OF THE INVENTION

In a first aspect, the present invention provides 1-isopropyl-4-{[4-(tetrahydro-2H-pyran-4-yloxy)phenyl]carbonyl}hexahydro-1H-1,4-diazepine mono-maleate.

This salt is the mono-maleate salt (the mono cis-butenedioic acid addition salt) of 1-isopropyl-4-{[4-(tetrahydro-2H-pyran-4-yloxy)phenyl]carbonyl}hexahydro-1H-1,4-diazepine, and has the following formula:

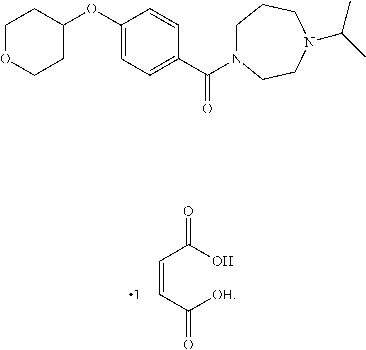

The invention also provides, in particular, 1-isopropyl-4-{[4-(tetrahydro-2H-pyran-4-yloxy)phenyl]carbonyl}hexahydro-1H-1,4-diazepine mono-maleate in a solid form, preferably in a crystalline form.

The invention also provides a pharmaceutical composition (e.g. adapted for oral administration) comprising 1-isopropyl-4-{[4-(tetrahydro-2H-pyran-4-yloxy)phenyl]carbonyl}hexahydro-1H-1,4-diazepine mono-maleate (e.g. in a crystalline form) and a pharmaceutically acceptable carrier.

In a second aspect, the present invention provides crystalline Form 1 of 1-isopropyl-4-{[4-(tetrahydro-2H-pyran-4-yloxy)phenyl]carbonyl}hexahydro-1H-1,4-diazepine mono-maleate.

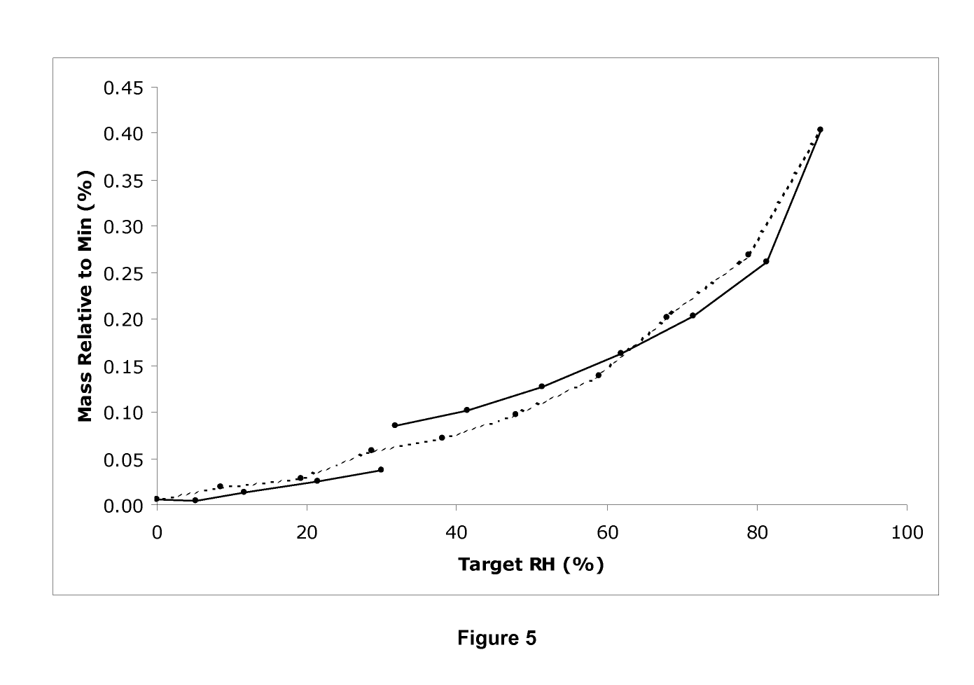

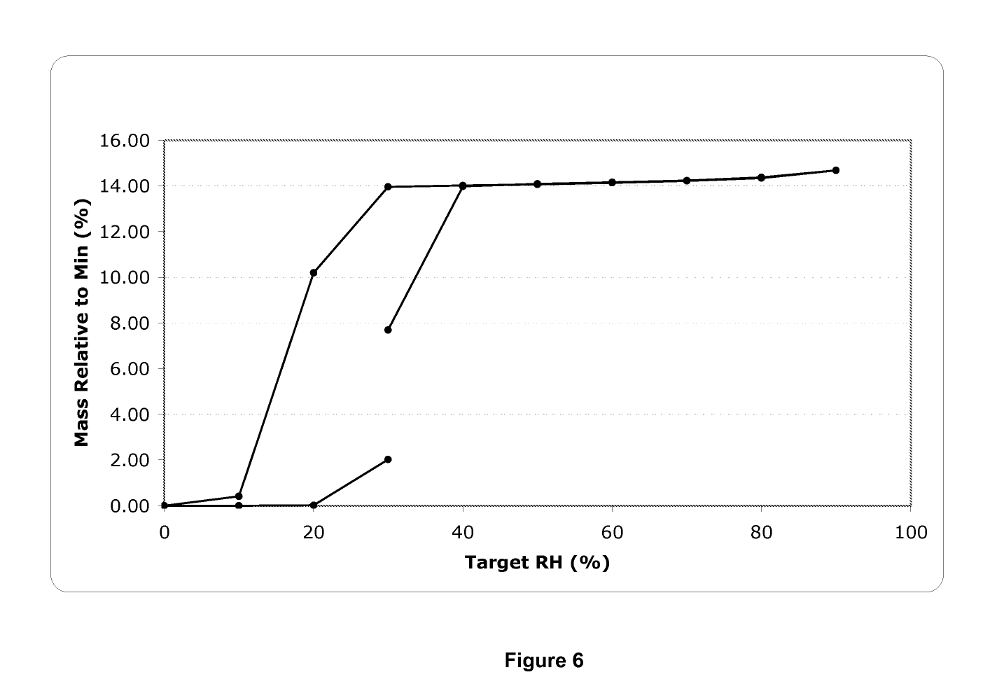

Gravimetric vapour sorption (GVS) analysis, which measures water sorption/desorption at different relative humidities, shows that crystalline Form 1 of 1-isopropyl-4-{[4-(tetrahydro-2H-pyran-4-yloxy)phenyl]carbonyl}hexahydro-1H-1,4-diazepine mono-maleate, reversibly adsorbs and/or absorbs, and/or desorbs, approximately 0.4% w/w water across the 0-90% relative humidity range at 25° C. under nitrogen (see FIG. 5 and Examples 4 and 5.3 for details). This represents an advantage, in respect of drug developability, over the “kinetic solid” 1-isopropyl-4-{[4-(tetrahydro-2H-pyran-4-yloxy)phenyl]carbonyl}hexahydro-1H-1,4-diazepine hydrochloride prepared in Example 2 Part C (the sample before purification by recrystallisation), which adsorbs and/or absorbs, and/or desorbs, higher quantities of water across the 0-90% relative humidity range at 25° C. under nitrogen (see FIG. 6 and Example 5.4 for details of comparative data).

Crystalline Form 1 of 1-isopropyl-4-{[4-(tetrahydro-2H-pyran-4-yloxy)phenyl]carbonyl}hexahydro-1H-1,4-diazepine mono-maleate can be characterized by its spectroscopic and/or physical characteristics, such as by its X-ray powder diffraction (XRPD) diffractogram (defined in the third and fourth aspects of the invention hereinbelow), by its solid-form infrared (IR) spectrum (defined in the fifth and sixth aspects of the invention hereinbelow), and/or by its differential scanning calorimetry (DSC) thermogram (defined in the seventh aspect of the invention hereinbelow).

Therefore, in a third aspect, the present invention provides crystalline 1-isopropyl-4-{[4-(tetrahydro-2H-pyran-4-yloxy)phenyl]carbonyl}hexahydro-1H-1,4-diazepine mono-maleate having an X-ray powder diffraction (XRPD) diffractogram comprising four or more (or preferably five or more, more preferably six or more, or most preferably all) of the following peaks at substantially the following degrees two-theta (2θ) values:

9.2±0.1°, 13.4±0.1°, 17.0±0.1°, 18.5±0.1°, 19.8±0.1°, 21.3±0.1°, and 27.8±0.1°;
wherein the X-ray powder diffraction diffractogram is measured with a X-ray powder diffractometer using copper K-alpha X-radiation and a step size of 0.0167° two-theta or less.

Alternatively or additionally, in the third aspect, the crystalline 1-isopropyl-4-{[4-(tetrahydro-2H-pyran-4-yloxy)phenyl]carbonyl}hexahydro-1H-1,4-diazepine mono-maleate has an X-ray powder diffraction (XRPD) diffractogram comprising eight or more (or preferably ten or more, more preferably twelve or more, still more preferably fourteen or more, or most preferably all) of the following peaks at substantially the following degrees two-theta (2θ) values:

9.2±0.1°, 11.8±0.1°, 13.4±0.1°, 14.7±0.1°, 15.8±0.1°, 16.2±0.1°, 17.0±0.1°, 18.5±0.1°, 19.8±0.1°, 20.3±0.1°, 20.5±0.1°, 21.0±0.1°, 21.3±0.1°, 27.1±0.1°, 27.4±0.1°, 27.8±0.1°, 28.4±0.1°, and 30.2±0.1°;
wherein the X-ray powder diffraction diffractogram is measured with a X-ray powder diffractometer using copper K-alpha X-radiation and a step size of 0.0167° two-theta or less.

Alternatively or additionally, in the third aspect, the crystalline 1-isopropyl-4-{[4-(tetrahydro-2H-pyran-4-yloxy)phenyl]carbonyl}hexahydro-1H-1,4-diazepine mono-maleate has an X-ray powder diffraction diffractogram characterised by peak positions in degrees two-theta (2θ) and calculated d-spacings in Angstroms (Å) as shown in the table below, wherein the experimental error in the peak positions is approximately ±0.1° two-theta:

| | d-spacing/ |
|---|---|
| 2θ/° | Å |
| 9.2 | 9.6 |
| 11.8 | 7.5 |
| 13.4 | 6.6 |
| 14.7 | 6.0 |
| 15.8 | 5.6 |
| 16.2 | 5.5 |
| 17.0 | 5.2 |
| 18.5 | 4.8 |
| 19.8 | 4.5 |
| 20.3 | 4.4 |
| 20.5 | 4.3 |
| 21.0 | 4.2 |
| 21.3 | 4.2 |
| 27.1 | 3.3 |
| 27.4 | 3.3 |
| 27.8 | 3.2 |
| 28.4 | 3.1 |
| 30.2 | 3.0 |

and wherein the X-ray powder diffraction diffractogram is measured with a X-ray powder diffractometer using the following acquisition conditions: copper K-alpha X-radiation, a generator tension of 40 kV, a generator current of 45 mA, a start angle of 2.0° two-theta, an end angle of 40.0° two-theta, a step size of 0.0167° two-theta, a time per step of 31.750s, and wherein the sample (e.g. a few milligrams) is prepared by being mounted on a silicon wafer (zero background) plate, typically resulting in a layer (typically a thin layer) of powder. This data is taken from the XRPD data shown in Example 5.1 in respect of the crystalline Form 1 mono-maleate salt prepared in Example 4.

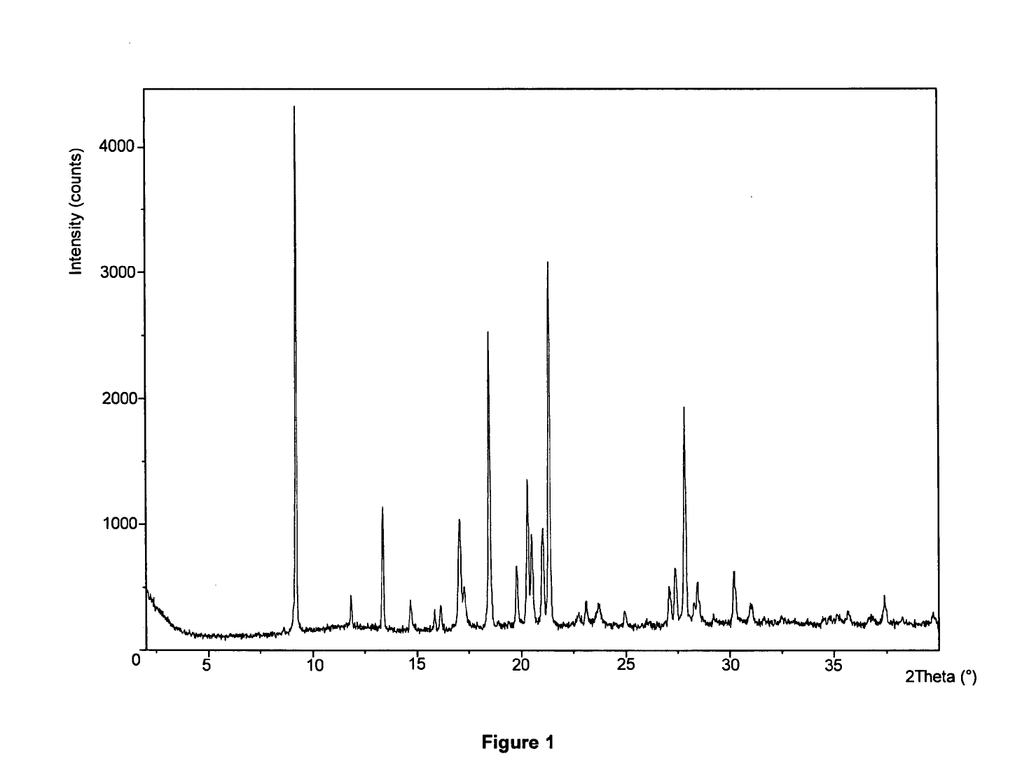

In a fourth aspect, the present invention provides crystalline 1-isopropyl-4-{[4-(tetrahydro-2H-pyran-4-yloxy)phenyl]carbonyl}hexahydro-1H-1,4-diazepine mono-maleate having an X-ray powder diffraction (XRPD) diffractogram substantially as shown in FIG. 1, wherein the X-ray powder diffraction diffractogram is measured with a X-ray powder diffractometer using copper K-alpha X-radiation and a step size of 0.0167° two-theta or less.

In the third and/or fourth aspects, the XRPD diffractogram is preferably measured using a time per step of 31.75 seconds or more. Additionally or alternatively, in the third and/or fourth aspects, the XRPD diffractogram is preferably measured using a sample mounted on a silicon wafer plate (preferably a silicon wafer zero background plate) and/or using a sample which is a layer (e.g. a thin layer) of powder.

In the third and/or fourth aspects, particularly in the fourth aspect, the XRPD diffractogram is preferably measured using a start angle of 2° two-theta (2θ) and an end angle of 40° two-theta (2θ).

In a fifth aspect, the present invention provides crystalline 1-isopropyl-4-{[4-(tetrahydro-2H-pyran-4-yloxy)phenyl]carbonyl}hexahydro-1H-1,4-diazepine mono-maleate having a solid-form attenuated total reflectance (ATR) infrared (IR) spectrum comprising five or more (or preferably six or more, more preferably seven or more, still more preferably eight or more, yet more preferably ten or more, or most preferably all) of the following peaks:

1700, 1622, 1464, 1422, 1353, 1247, 1234, 1089, 1048, 869, 840 and 765 cm⁻¹;
with a variation allowed for each peak of ±2 cm⁻¹.

Alternatively or additionally, in the fifth aspect, the crystalline 1-isopropyl-4-{[4-(tetrahydro-2H-pyran-4-yloxy)phenyl]carbonyl}hexahydro-1H-1,4-diazepine mono-maleate has a solid-form attenuated total reflectance (ATR) infrared (IR) spectrum comprising ten or more (or preferably fifteen or more, more preferably twenty or more, still more preferably thirty or more, yet more preferably thirty-five or more, or most preferably all) of the following peaks:

1700, 1622, 1604, 1575, 1509, 1464, 1422, 1393, 1375, 1353, 1341, 1308, 1297, 1280, 1247, 1234, 1205, 1178, 1169, 1153, 1132, 1115, 1089, 1069, 1048, 1017, 1005, 985, 962, 944, 908, 883, 869, 840, 828, 802, 784, 765, 725 and 685 cm⁻¹;
with a variation allowed for each peak of ±2 cm⁻¹.

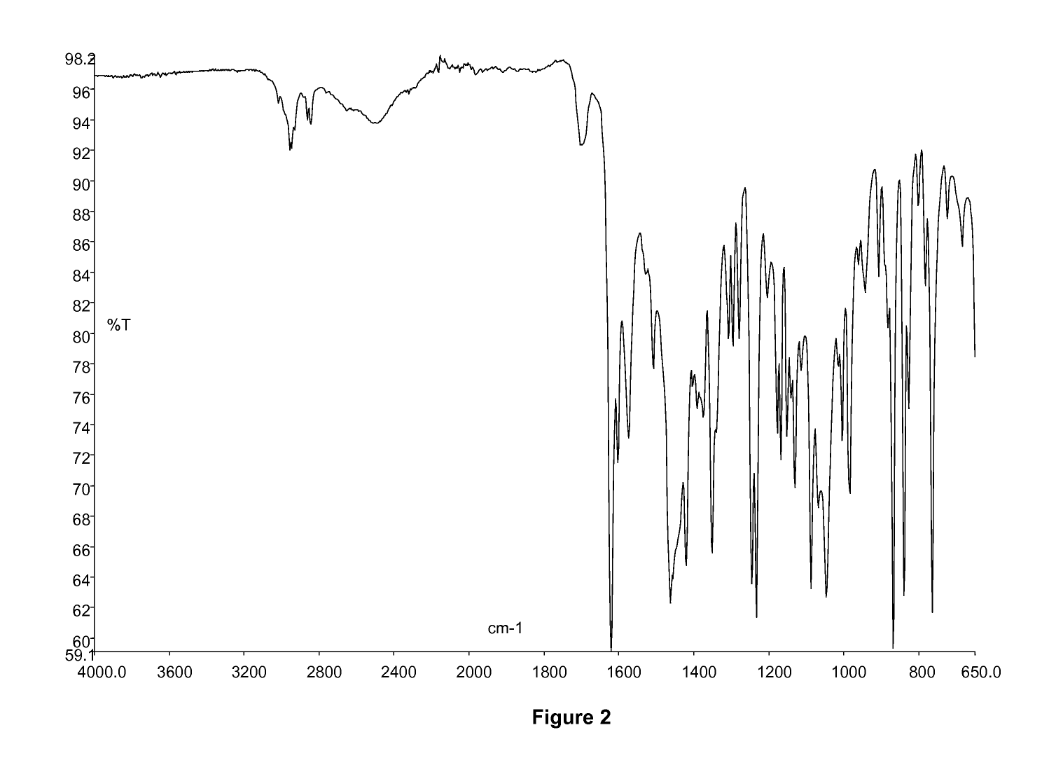

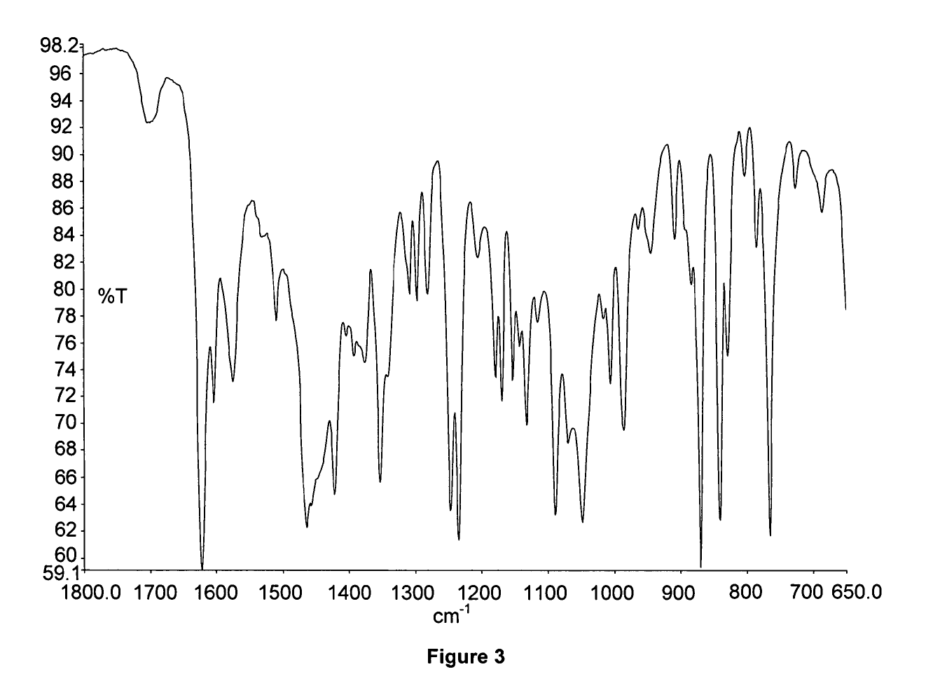

In any IR spectrum plotting percent transmittance on the vertical axis with high transmittance at the top of the spectrum (e.g., as in FIGS. 2 and 3), a vibrational mode or band causing an absorption of IR radiation will be shown as a down-pointing trough or valley (of lower transmittance) and not as an up-pointing peak. Therefore, the term “peak” or “band”, when used herein in respect of an IR spectrum, includes a down-pointing trough representing an absorption or decreased transmittance of IR radiation. This is as understood by the skilled person.

In a sixth aspect, the present invention provides crystalline 1-isopropyl-4-{[4-(tetrahydro-2H-pyran-4-yloxy)phenyl]carbonyl}hexahydro-1H-1,4-diazepine mono-maleate having a solid-form attenuated total reflectance (ATR) infrared (IR) spectrum substantially as shown in FIG. 2 and/or FIG. 3.

In the fifth and/or sixth aspects, the solid-form IR spectrum can for example be measured using an FT-IR (Fourier Transform Infrared) spectrometer, such as an FT-IR spectrometer fitted with an attenuated total reflectance (ATR) sampling accessory (e.g. a diamond/ZnSe ATR sampling accessory), and/or can for example be measured at 4 cm⁻¹ resolution.

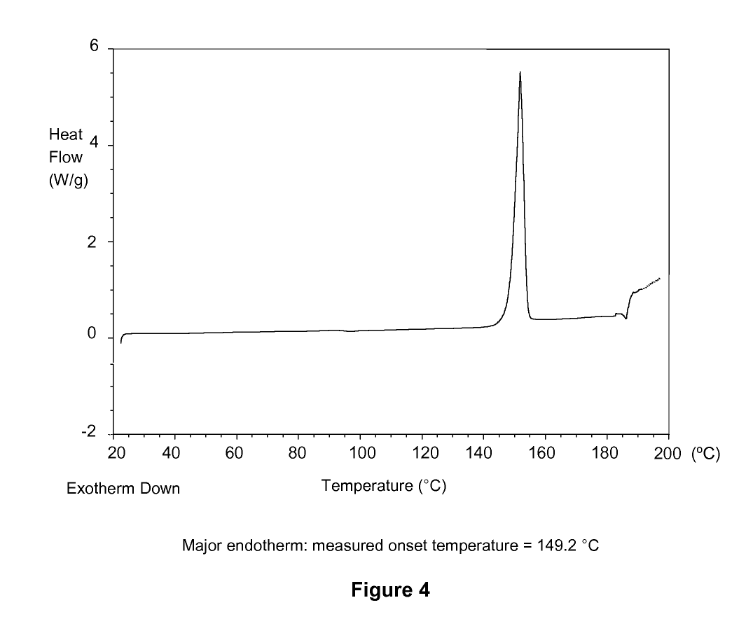

In a seventh aspect, the present invention provides crystalline 1-isopropyl-4-{[4-(tetrahydro-2H-pyran-4-yloxy)phenyl]carbonyl}hexahydro-1H-1,4-diazepine mono-maleate having a differential scanning calorimetry (DSC) thermogram which is substantially as shown in FIG. 4 and/or which has an endotherm with an onset temperature of 149.2±5° C. (or, more particularly, 149.2±1.5° C.), when measured at atmospheric pressure and under a flow of nitrogen using a calorimeter using a heating rate of about 10° C. per minute.

The onset temperature of a DSC endotherm is calculated as the temperature at which a linear extrapolation of the up-curve of the endotherm (i.e. of the lower-temperature portion of the endotherm, e.g. of the left-hand portion of the endotherm in FIG. 4) meets the baseline of the DSC thermogram.

The seventh aspect of the present invention also provides crystalline 1-isopropyl-4-{[4-(tetrahydro-2H-pyran-4-yloxy)phenyl]carbonyl}hexahydro-1H-1,4-diazepine mono-maleate having a melt onset temperature of 149.2±5° C. (or, more particularly, 149.2±1.5° C.). Preferably the melt onset temperature can be when measured using differential scanning calorimetry (DSC), e.g. at atmospheric pressure and under a flow of nitrogen using a calorimeter using a heating rate of about 10° C. per minute.

In an eighth aspect, the present invention provides a process for the preparation of crystalline Form 1 of 1-isopropyl-4-{[4-(tetrahydro-2H-pyran-4-yloxy)phenyl]carbonyl}hexahydro-1H-1,4-diazepine mono-maleate, comprising:

(a) mixing 1-isopropyl-4-{[4-(tetrahydro-2H-pyran-4-yloxy)phenyl]carbonyl}hexahydro-1H-1,4-diazepine, a solvent comprising (e.g. consisting essentially of) a C₂₋₄alkyl C₂₋₄alkanoate (e.g. in particular a C₂₋₄alkyl acetate such as ethyl acetate, isopropyl acetate or n-butyl acetate; or preferably a solvent comprising, e.g. consisting essentially of, ethyl acetate), and maleic acid (cis-butenedioic acid, preferably about 1 mole equivalent thereof with respect to the molar amount of 1-isopropyl-4-{[4-(tetrahydro-2H-pyran-4-yloxy)phenyl]carbonyl}hexahydro-1H-1,4-diazepine), under conditions in which the maleic acid and the 1-isopropyl-4-{[4-(tetrahydro-2H-pyran-4-yloxy)phenyl]carbonyl}hexahydro-1H-1,4-diazepine are dissolved in the solvent,
(b) optionally (and preferably), seeding the resulting mixture with crystalline Form 1 of 1-isopropyl-4-{[4-(tetrahydro-2H-pyran-4-yloxy)phenyl]carbonyl}hexahydro-1H-1,4-diazepine mono-maleate,
(c) allowing or causing the crystalline Form 1 of 1-isopropyl-4-{[4-(tetrahydro-2H-pyran-4-yloxy)phenyl]carbonyl}hexahydro-1H-1,4-diazepine mono-maleate to crystallise from the mixture (e.g. using temperature cycling such as temperature cycling within the range of about 0-40° C., and/or by stirring or resting the mixture for four or more hours e.g. at a temperature of about 0-40° C. such as at a temperature of from about 0° C. to room temperature),
(d) separating (in particular by filtration) the crystalline Form 1 of 1-isopropyl-4-{[4-(tetrahydro-2H-pyran-4-yloxy)phenyl]carbonyl}hexahydro-1H-1,4-diazepine mono-maleate from the solvent, and
(e) optionally (and preferably), drying (in particular, under vacuum, and/or at about 35-60° C. such as about 40° C., and/or for from 0.5 to 3 days) the crystalline Form 1 of 1-isopropyl-4-{[4-(tetrahydro-2H-pyran-4-yloxy)phenyl]carbonyl}hexahydro-1H-1,4-diazepine mono-maleate.

In a ninth aspect, the present invention provides a process for the preparation of 1-isopropyl-4-{[4-(tetrahydro-2H-pyran-4-yloxy)phenyl]carbonyl}hexahydro-1H-1,4-diazepine

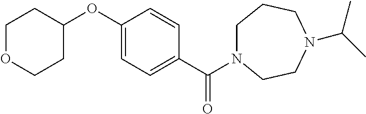

or a pharmaceutically acceptable salt thereof, such as the hydrochloride (typically monohydrochloride) or maleate (typically mono-maleate) salt thereof, which process comprises:
(a) reacting a non-acid-chloride derivative of 4-(tetrahydro-2H-pyran-4-yloxy)benzoic acid, in which the carboxylic acid group has been activated, with 1-isopropyl-hexahydro-1H-1,4-diazepine;
and (b) optionally preparing a pharmaceutically acceptable salt (e.g. hydrochloride salt or maleate (typically mono-maleate) salt) of 1-isopropyl-4-{[4-(tetrahydro-2H-pyran-4-yloxy)phenyl]carbonyl}hexahydro-1H-1,4-diazepine

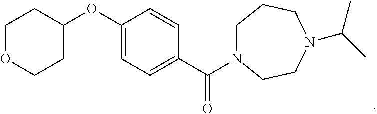

Process (a) typically comprises activation of 4-(tetrahydro-2H-pyran-4-yloxy)benzoic acid with a coupling reagent (e.g. in a suitable solvent e.g. a polar aprotic organic solvent, such as acetonitrile, propionitrile, N,N-dimethylformamide or dimethylsulfoxide), followed by reaction with 1-isopropyl-hexahydro-1H-1,4-diazepine.

In one embodiment, the coupling reagent is an organic di-substituted carbodiimide, such as 1-(3-dimethylaminopropyl)-3-ethylcarbodiimide hydrochloride (EDC) in which case: the reaction can optionally be carried out in the presence of 1-hydroxybenzotriazole (HOBT), and/or the reaction solvent can for example be N,N-dimethylformamide, and/or the reaction temperature can e.g. be from about 0° C. to about 40° C., such as room temperature.

In an alternative particular embodiment, the coupling reagent is carbonyl diimidazole or pivaloyl chloride (trimethylacetyl chloride).

Most preferably, the coupling reagent is carbonyl diimidazole. For example in a medium or large scale process, the use of carbonyl diimidazole (CDI) as coupling reagent gives reasonably good yields and/or a reasonably clean reaction, as well as being less expensive than the 1-(3-dimethylaminopropyl)-3-ethylcarbodiimide hydrochloride coupling agent.

In process (a), preferably, the activation of the 4-(tetrahydro-2H-pyran-4-yloxy)benzoic acid with the carbonyl diimidazole coupling reagent, and the subsequent reaction with 1-isopropyl-hexahydro-1H-1,4-diazepine, are both carried out in a reaction solvent comprising (or, in one particular embodiment, consisting essentially of) acetonitrile and/or propionitrile, more preferably acetonitrile.

When carbonyl diimidazole (CDI) is used as coupling reagent for activation of the 4-(tetrahydro-2H-pyran-4-yloxy)benzoic acid, followed by reaction with the 1-isopropyl-hexahydro-1H-1,4-diazepine, then the reaction conditions can in particular be as follows, independently and/or in any combination:
the carbonyl diimidazole is typically present in 0.5 to 1.5 mole equivalents (with reference to the number of moles of the 4-(tetrahydro-2H-pyran-4-yloxy)benzoic acid), suitably 0.9 to 1.1 mole equivalents, preferably 1.0 to 1.1 mole equivalents, e.g. 1.1 mole equivalents; and/or
the 1-isopropyl-hexahydro-1H-1,4-diazepine is typically present in 0.5 to 1.5 mole equivalents (with reference to the number of moles of the 4-(tetrahydro-2H-pyran-4-yloxy)benzoic acid), suitably 1.0 to 1.25 mole equivalents, preferably 1.1 to 1.2 mole equivalents, e.g. 1.15 or 1.2 mole equivalents; and/or
the reaction (the activation of the 4-(tetrahydro-2H-pyran-4-yloxy)benzoic acid with CDI, or the subsequent reaction with 1-isopropyl-hexahydro-1H-1,4-diazepine, or both) is typically carried out in a suitable organic solvent such as a polar aprotic organic solvent, for example a solvent comprising (e.g. consisting essentially of) acetonitrile, propionitrile, dimethylsulfoxide, N,N-dimethylformamide (DMF), N-methylpyrrolidinone (NMP), and/or 1,4-dioxane; preferably the reaction solvent comprises or consists essentially of acetonitrile and/or propionitrile, more preferably acetonitrile; and/or
the reaction solvent is typically dry, although a small percentage of water in the reaction solvent can sometimes be tolerated; and/or
when the reaction solvent is acetonitrile or propionitrile, the temperature of the reaction (for either the activation of the 4-(tetrahydro-2H-pyran-4-yloxy)benzoic acid with the carbonyl diimidazole, or for the subsequent reaction with the 1-isopropyl-hexahydro-1H-1,4-diazepine, or for both) can for example be from about 0° C. to the boiling point or reflux temperature of the solvent. The temperature of the activation reaction can e.g. be in the range of about 20 to about 40° C. (e.g. about 30° C.), e.g. followed by reaction with the 1-isopropyl-hexahydro-1H-1,4-diazepine at a temperature of from about 20° C. to the boiling point or reflux temperature of the reaction solvent (e.g. from about 40 to about 60° C., e.g. about 50° C.); this low activation reaction temperature can potentially help to maximise yield due to decreased CDI decomposition, but any surviving excess CDI after the activation reaction is more likely to react with the later-added 1-isopropyl-hexahydro-1H-1,4-diazepine to form an impurity. Hence, it is currently thought preferable to activate the 4-(tetrahydro-2H-pyran-4-yloxy)benzoic acid with the carbonyl diimidazole at a temperature of from about 50° C. to the boiling point/reflux temperature of the reaction solvent or from about 60° C. to the boiling/reflux temperature (e.g. about 60 to about 70° C., e.g. 65 to 70° C., e.g. in acetonitrile solvent), and optionally also to have this temperature range (from about 50° C. to the boiling point/reflux temperature, e.g. about 60 to about 70° C.) as the temperature for the subsequent reaction with the 1-isopropyl-hexahydro-1H-1,4-diazepine; and/or
the 4-(tetrahydro-2H-pyran-4-yloxy)benzoic acid and the carbonyl diimidazole are typically reacted together (e.g. with stirring) for at least 0.5 hours, suitably for at least 2 hours, e.g. for 0.5 to 5 hours such as 0.5 to 3 hours, e.g. for 2 to 5 hours or 2 to 3 hours, before the 1-isopropyl-hexahydro-1H-1,4-diazepine is mixed with the activated 4-(tetrahydro-2H-pyran-4-yloxy)benzoic acid; and/or
the product of activation of the 4-(tetrahydro-2H-pyran-4-yloxy)benzoic acid by the carbonyl diimidazole, and the 1-isopropyl-hexahydro-1H-1,4-diazepine, are typically reacted together (e.g. with stirring) for at least 0.5 hours (e.g. 0.5 to 24 hours), suitably for at least 1 hour (e.g. 1 to 24 hours, e.g. 1 to 3 hours or 10 to 24 hours), such as for at least 2 hours (e.g. 2 to 24 hours, e.g. 2 to 3 hours or 10 to 24 hours).

In the ninth aspect of the invention, preferably, the 1-isopropyl-hexahydro-1H-1,4-diazepine used in step (a) is prepared by treating 1-isopropyl-hexahydro-1H-1,4-diazepine bis-trifluoroacetate with a base (e.g. aqueous base, e.g. aqueous sodium or potassium hydroxide solution). This can be carried out in particular by dissolving the 1-isopropyl-hexahydro-1H-1,4-diazepine bis-trifluoroacetate in a base (e.g. aqueous sodium or potassium hydroxide solution, e.g. ca. 1M to ca. 5M such as ca. 2M), extracting the aqueous mixture with a non-water-miscible organic extraction solvent (e.g. dichloromethane) one or more times, separating the organic extraction solvent layer(s) (the organic extract(s)), optionally removing water from the organic extract(s), and then removing the organic extraction solvent from the organic extract (e.g. by evaporation or by distillation) to isolate the 1-isopropyl-hexahydro-1H-1,4-diazepine.

Using 1-isopropyl-hexahydro-1H-1,4-diazepine bis-trifluoroacetate, as the starting material to prepare the free base 1-isopropyl-hexahydro-1H-1,4-diazepine, has certain advantages over the use of 1-(isopropyl)-hexahydro-1H-1,4-diazepine dihydrochloride which was disclosed in the synthesis of Example 10 of WO 2005/040144 A1, specifically:

(i) The use of 1-isopropyl-hexahydro-1H-1,4-diazepine bis-trifluoroacetate allows the avoidance of the use of toxic dioxane in the preparation of 1-(isopropyl)-hexahydro-1H-1,4-diazepine dihydrochloride (from the BOC-protected precursor e.g. Intermediate 1), which preparation typically (see e.g. Intermediate 2 herein) uses HCl (e.g. 4M HCl) in dry dioxane (to avoid the water present in aqueous hydrochloric acid) in order to obtain solid 1-(isopropyl)hexahydro-1H-1,4-diazepine dihydrochloride.
(ii) Importantly, the solid 1-(isopropyl)hexahydro-1H-1,4-diazepine dihydrochloride is very hygroscopic, and forms a gum if left in air at normal humidities (normal in the United Kingdom). This hygroscopicity makes it difficult to quantify and weigh the dihydrochloride salt for the coupling reaction, and also can lead to the presence of undesired water in the coupling reaction (e.g. with DCI).
(iii) The 1-isopropyl-hexahydro-1H-1,4-diazepine bis-trifluoroacetate is a crystalline solid, appears to have long-term stability, is free-flowing, and most importantly it is substantially non-hygroscopic at normal relative humidity (e.g. ca. 30%) at 25° C.

Hence, in the process of the ninth aspect of the invention, the use of solid 1-isopropyl-hexahydro-1H-1,4-diazepine bis-trifluoroacetate salt has an advantage with respect to storage, handling and/or processing, and/or potentially giving a better water-free coupling (e.g. DCI-coupling) reaction, compared to the use of 1-(isopropyl)-hexahydro-1H-1,4-diazepine dihydrochloride.

Therefore, in a tenth aspect of the present invention, there is provided 1-isopropyl-hexahydro-1H-1,4-diazepine bis-trifluoroacetate (e.g. in solid such as crystalline form), having the following formula:

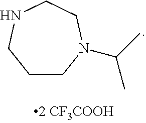

The tenth aspect of the invention also provides the use of 1-isopropyl-hexahydro-1H-1,4-diazepine bis-trifluoroacetate (e.g. in solid such as crystalline form) as a chemical intermediate, in particular in the preparation of 1-isopropyl-4-{[4-(tetrahydro-2H-pyran-4-yloxy)phenyl]carbonyl}hexahydro-1H-1,4-diazepine or a pharmaceutically acceptable salt (e.g. mono-maleate salt) thereof.

The 1-isopropyl-hexahydro-1H-1,4-diazepine bis-trifluoroacetate can be prepared by the following reaction:

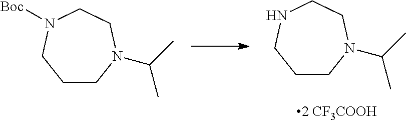

wherein the reaction comprises:
(i) reaction of the 1-tert-butyl-4-isopropyl-hexahydro-1H-1,4-diazepine-1-carboxylate (e.g. see Intermediate 1 for preparations) with trifluoroacetic acid (e.g. 2-20 mole equivalents, e.g. ca. 10 mole equivalents) in a suitable non-aqueous organic solvent (e.g. dichloromethane), e.g. at 20-40° C. such as 27-33° C. and/or e.g. for 6 to 48 hours such as for 12 to 18 hours, and
(ii) evaporation of the resulting mixture to dryness, and
(iii) precipitation (e.g. crystallisation) from a suitable non-aqueous organic precipitation solvent (e.g. tert-butyl methyl ether optionally also with a minor amount (e.g. 2-10% e.g. 3-5% by volume of solvent) of ethyl acetate).

See for example Intermediate 3 hereinafter.

General process for preparation of the hydrochloride salt (typically monohydrochloride salt) of 1-isopropyl-4-{[4-(tetrahydro-2H-pyran-4-yloxy)phenyl]carbonyl}hexahydro-1H-1,4-diazepin

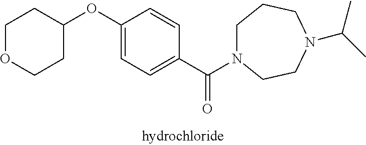

To prepare, crystallise and isolate a hydrochloride salt (e.g. monohydrochloride) of the 1-isopropyl-4-{[4-(tetrahydro-2H-pyran-4-yloxy)phenyl]carbonyl}hexahydro-1H-1,4-diazepine, in one embodiment, at the end of the reaction in which the 4-(tetrahydro-2H-pyran-4-yloxy)benzoic acid has been activated by a coupling reagent (e.g. carbonyl diimidazole) followed by reaction of the activated acid with the 1-isopropyl-hexahydro-1H-1,4-diazepine, in a reaction solvent such as acetonitrile or propionitrile, the following process can be carried out:
typically, the volume of reaction solvent (e.g. propionitrile or preferably acetonitrile) is reduced under reduced pressure, e.g. to about 2-4 volumes e.g. about 2.5-3 volumes (e.g. of propionitrile or preferably acetonitrile); and then, optionally, about 2-6 volumes (e.g. about 2-4 or about 5-6 volumes, e.g. ca. 3 volumes) of isopropanol is added and the volume of solvent is reduced under reduced pressure e.g. to about 2-4 volumes e.g. about 2.5-3 volumes of solvent; and
then a solution of HCl in a suitable solvent (e.g. a crystallisation solvent as defined below e.g. isopropanol; e.g. ca. 5-6 N HCl in isopropanol such as ca. 0.7-1.1 volumes e.g. ca. 0.9 volumes thereof) is added to the reaction mixture; with preferably the HCl being added in an amount of 0.5 to 1.3 mole equivalents such as 0.85 to 1.05 mole equivalents e.g. 1.0 mole equivalents with respect to the molar amount of the 4-(tetrahydro-2H-pyran-4-yloxy)benzoic acid starting material used; and
preferably, before and/or after and/or at the same time as the addition of the appropriate pharmaceutically acceptable salt-forming acid (e.g. HCl), a crystallisation solvent is added (wherein the crystallisation solvent can e.g. comprise or be: an alcohol being a C₁₋₃ alcohol or n-butanol (including mixtures of alcohols), for example isopropanol, n-propanol, n-butanol, ethanol, or methanol; a mixture of water and an alcohol being a C₁₋₃ alcohol or n-butanol, for example isopropanol:water, ethanol:water, or methanol:water; isopropyl acetate; ethyl acetate; a C₃₋₆ ketone such as methyl isobutyl ketone (MIBK), methyl ethyl ketone, or acetone; acetonitrile; or dichloromethane; and wherein suitably the crystallisation solvent comprises or is an alcohol being a C₁₋₃ alcohol or n-butanol (including mixtures of alcohols), or a mixture of water and an alcohol being a C₁₋₃ alcohol or n-butanol; such as preferably: isopropanol, isopropanol:water (in particular ca. 2-10% water in isopropanol, e.g. ca. 3-7% or ca. 2-5% water in isopropanol, such as ca. 3-5% or ca. 4-5% water in isopropanol), or ethanol:water such as ca. 1-5% water in ethanol or industrial methylated spirits); and for example
as a typical example of the addition of the crystallisation solvent: 6 to 12 volumes, e.g. about 6-10 volumes, e.g. about 9-10 volumes of the crystallisation solvent (e.g. as herein defined, e.g. isopropanol or ca. 2-10% water in isopropanol, e.g. ca. 3-7% or ca. 2-5% such as ca. 4-5% water in isopropanol) can be added, before and/or after and/or at the same time as the addition of the HCl;
   and
preferably, the solvent-containing mixture comprising the hydrochloride salt product is at, or is heated to, a temperature of about 50° C. to the boiling point or reflux temperature of the solvent (e.g. about 50-75° C., e.g. about 60-70° C., e.g. about 60-65° C.), and
the hydrochloride salt (e.g. monohydrochloride salt) of the 1-isopropyl-4-{[4-(tetrahydro-2H-pyran-4-yloxy)phenyl]carbonyl}hexahydro-1H-1,4-diazepine is allowed or caused to crystallise or recrystallise from the hot mixture (e.g. by cooling the hot mixture, e.g. by cooling it to ca. 17-35° C., such as by cooling it to ca. 25-35° C. such as ca. 30° C. and/or by cooling it to room temperature (typically ca. 17-25° C.), and if by cooling then preferably by gradual cooling of the hot mixture over a period of 1-3 hours or more; and optionally by stirring the cooled mixture for ca. 5-48 hours e.g. ca. 10-30 hours), and
the crystalline hydrochloride salt (e.g. monohydrochloride salt) of the 1-isopropyl-4-{[4-(tetrahydro-2H-pyran-4-yloxy)phenyl]carbonyl}hexahydro-1H-1,4-diazepine is isolated from the solvent (e.g. by filtration), and is usually dried (e.g. by drying under reduced pressure at about 40-60° C. such as about 50° C. (e.g. vacuum oven drying), or e.g. by drying at room temperature e.g. under suction or a stream of gas such as air or nitrogen).

As used in this section, “volumes” of solvents/solutions/liquids are stated with respect to 4-(tetrahydro-2H-pyran-4-yloxy)benzoic acid, equating 1 ml of the former to 1 g of the latter. For example: if 10 g of 4-(tetrahydro-2H-pyran-4-yloxy)benzoic acid is used, then 0.9 volumes of 5-6N HCl in isopropanol means 0.9×10=9 ml of 5-6N HCl in isopropanol.

General process for preparation and/or isolation of 1-isopropyl-4-{[4-(tetrahydro-2H-pyran-4-yloxy)phenyl]carbonyl}hexahydro-1H-1,4-diazepine (the “free base”)

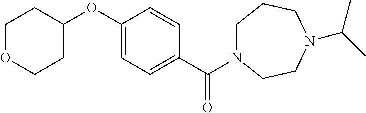

To prepare and/or isolate 1-isopropyl-4-{[4-(tetrahydro-2H-pyran-4-yloxy)phenyl]carbonyl}hexahydro-1H-1,4-diazepine, the “free base”, from a or the reaction mixture:
the reaction solvent can be removed; and/or
the free base can be dissolved in isopropyl acetate and then crystallised by adding heptane to the isopropyl acetate solution—e.g. the free base can be crystallised from about 1:2 isopropyl acetate:heptane; and/or
after crystallisation, the free base can be separated (e.g. by filtration) and dried (e.g. by drying under reduced pressure at about 40-60° C. such as about 50° C. (e.g. vacuum oven drying), e.g. overnight) to give solid 1-isopropyl-4-{[4-(tetrahydro-2H-pyran-4-yloxy)phenyl]carbonyl}hexahydro-1H-1,4-diazepine (the “free base”).

In one particular embodiment, the isolated solid free base is recrystallised, preferably recrystallised from tert-butyl methyl ether (e.g. using about 10-20 volumes, preferably about 15 volumes, of tert-butyl methyl ether, with respect to the weight of free base to be recrystallised). For example, the free base can be recrystallised from tert-butyl methyl ether by dissolving the free base in tert-butyl methyl ether (e.g. about 10-20 volumes, preferably about 15 volumes, thereof, with respect to the weight of free base to be recrystallised) at about 40-60° C. e.g. about 50° C., allowing the solution to cool gradually to room temperature to give a slurry, separating the solid free base from the slurry (e.g. by filtration), and drying the separated solid (e.g. in a vacuum oven overnight at about 40-60° C. e.g. about 50° C.), to give 1-isopropyl-4-{[4-(tetrahydro-2H-pyran-4-yloxy)phenyl]carbonyl}hexahydro-1H-1,4-diazepine as a solid.

For preparation of 1-isopropyl-4-{[4-(tetrahydro-2H-pyran-4-yloxy)phenyl]carbonyl}hexahydro-1H-1,4-diazepine mono-maleate, specifically crystalline Form 1 thereof, from the free base, see for example the eighth aspect of the present invention hereinabove, and see for example Examples 3 and 4 hereinafter.

### Preparation of Intermediates

4-(Tetrahydro-2H-pyran-4-yloxy)benzoic acid (IV), which is used as an intermediate in the ninth aspect of the present invention, may be prepared in accordance with the following scheme wherein P represents a suitable protecting group, such as C₁₋₆ straight-chain alkyl (e.g. methyl, ethyl, n-propyl or n-butyl) or isopropyl or isobutyl, or benzyl; such as methyl or ethyl; in particular methyl.

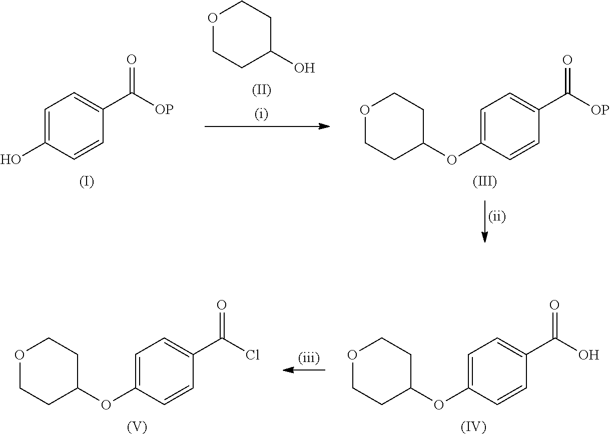

Step (i) typically comprises the use of a phosphine such as triphenylphosphine in a suitable solvent such as tetrahydrofuran, toluene and/or xylene (wherein “xylene” can be o-xylene, m-xylene, p-xylene, or a mixture of xylenes), followed by the addition (e.g. slow and/or dropwise addition) of an azodicarboxylate such as diethyl azodicarboxylate or diisopropyl azodicarboxylate, at a suitable temperature, for example, from room temperature to about 80° C., e.g. room temperature. Reaction times (including any azodicarboxylate addition time) can be e.g. from 0.5 to 72 hours. When using tetrahydrofuran as reaction solvent, room temperature can be used, and the reaction time is for example from 3 to 72 hours. When the reaction solvent comprises or consists essentially of toluene and/or xylene, in particular toluene, a reaction temperature of about 40 to about 80° C., e.g. about 40 to about 70° C., e.g. about 55° C., can be used; and/or a reaction time (including any azodicarboxylate addition time) of about 0.5 to 6 hours, e.g. 0.5 to 3 hours, e.g. 1-2 hours, can be used.

In a particular embodiment, the step (i) reaction solvent comprises or consists essentially of toluene and/or xylene, preferably toluene, and reaction step (i) is carried out in the presence of triphenylphosphine and diisopropyl azodicarboxylate (e.g. ca. 1.3-2.0 e.g. ca. 1.5 mole equivalents of triphenylphosphine and/or diisopropyl azodicarboxylate with reference to compound (I); and/or e.g. the triphenylphosphine and the diisopropyl azodicarboxylate are present in substantially the same number of mole equivalents as each other); in which case suitably the heated (e.g. ca. 40-70° C.) reaction mixture can be cooled (e.g. to −10 to 25° C., e.g. to ca. 0-5° C., provided that it is not cooled to the melting point of the solvent or below), e.g. for 0.5 to 2 hours, and then the solid biproduct formed is removed e.g. by filtration. The use of toluene as reaction solvent helps to crystallise out the biproduct adduct of triphenylphosphine oxide and diisopropyl hydrazinedicarboxylate from the solution (especially when the reaction mixture is seeded with this adduct e.g. after cooling), which helps to reduce the levels of triphenylphosphine oxide in the crude product (III).

When using toluene and/or xylene as a solvent in reaction step (i), in one embodiment, the reaction product compound of formula (III) is not isolated. Optionally, in this embodiment, the toluene and/or xylene solution of the compound of formula (III) is used directly in the subsequent reaction (deprotection e.g. hydrolysis) step (ii), in particular when C₁₋₆ straight-chain alkyl (e.g. methyl, ethyl, n-propyl or n-butyl) or isopropyl or isobutyl and the subsequent step (ii) comprises alkaline (e.g. NaOH or KOH) hydrolysis of the ester.

According to a eleventh aspect of the invention, there is provided a process for preparing a compound of formula (III)

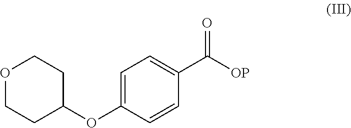

wherein P represents a protecting group [such as C₁₋₆ straight-chain alkyl (e.g. methyl, ethyl, n-propyl or n-butyl) or isopropyl or isobutyl, or benzyl; in particular C₁₋₆ straight-chain alkyl or isopropyl, e.g. methyl or ethyl], wherein the process comprises:
(i) reacting the compound of formula (I)

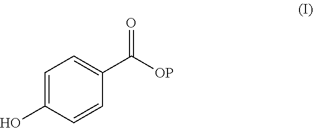

, wherein P represents the protecting group as defined for the compound of formula (III), with 4-hydroxytetrahydropyran of formula (II) or a derivative thereof in which its OH group is activated;
wherein the reaction step (i) is carried out in a reaction solvent comprising or consisting essentially of toluene and/or xylene (in particular toluene).

“Xylene” can be o-xylene, m-xylene, p-xylene, or a mixture of xylenes.

In this eleventh aspect of the invention using a step (i) reaction solvent comprising toluene and/or xylene, the reaction conditions for step (i) can in particular be as described herein for step (i) for the Preparation of Intermediates.

Preferably, in this eleventh aspect, reaction step (i) is carried out in the presence of triphenylphosphine and diisopropyl azodicarboxylate (e.g. ca. 1.3-2.0 e.g. ca. 1.5 mole equivalents of triphenylphosphine and/or diisopropyl azodicarboxylate with reference to compound (I); and/or e.g. the triphenylphosphine and the diisopropyl azodicarboxylate present in substantially the same number of mole equivalents as each other). For a step (i) reaction solvent comprising toluene and/or xylene, in particular toluene, a reaction temperature of about 40 to about 80° C., e.g. about 40 to about 70° C., e.g. about 55° C., can be used; and/or a reaction time (including any azodicarboxylate addition time) of about 0.5 to 6 hours, e.g. 0.5 to 3 hours, e.g. 1-2 hours, can be used in step (i).

In a particular embodiment of this eleventh aspect, when the step (i) reaction solvent comprises toluene and/or xylene, preferably toluene, and reaction step (i) is carried out in the presence of triphenylphosphine and diisopropyl azodicarboxylate, the heated (e.g. ca. 40-80° C., e.g. ca. 40-70° C.) reaction mixture can be cooled (e.g. to −10 to 25° C., e.g. to ca. 0-5° C., provided that it is not cooled to the melting point of the solvent or below), e.g. for 0.5 to 2 hours, and then the solid biproduct formed (the adduct of triphenylphosphine oxide and diisopropyl hydrazinedicarboxylate) is removed e.g. by filtration. In particular, the reaction mixture can be seeded with the adduct of triphenylphosphine oxide and diisopropyl hydrazinedicarboxylate, e.g. after cooling the reaction mixture. The use of toluene as reaction solvent helps to crystallise out the biproduct adduct of triphenylphosphine oxide and diisopropyl hydrazinedicarboxylate from the solution (especially when the reaction mixture is seeded with this adduct e.g. after cooling), which helps to reduce the levels of triphenylphosphine oxide in the crude product (III).

For the eleventh aspect of the invention, being the process for preparing a compound of formula (III) using a step (i) reaction solvent comprising toluene and/or xylene, there is also provided a process for preparing a compound of formula (IV), which is 4-(tetrahydro-2H-pyran-4-yloxy)benzoic acid, wherein the process comprises:
performing reaction step (i) using a reaction solvent comprising or consisting essentially of toluene and/or xylene (e.g. as described herein), and then
(ii) converting the resulting compound of formula (III) to the compound of formula (IV); e.g. by hydrolysing the ester within the compound of formula (III) when P represents C₁₋₆ straight-chain alkyl (e.g. methyl, ethyl, n-propyl or n-butyl) or isopropyl or isobutyl (in particular methyl or ethyl), e.g. under alkaline conditions (e.g. using sodium hydroxide or potassium hydroxide, e.g. aqueous); or e.g. by hydrogenation when P represents benzyl.

In a twelfth aspect of the present invention, there is provided a process for the preparation of 1-isopropyl-4-{[4-(tetrahydro-2H-pyran-4-yloxy)phenyl]carbonyl}hexahydro-1H-1,4-diazepine

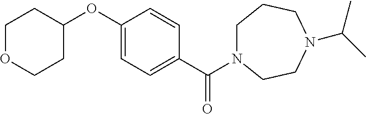

or a salt thereof (e.g. pharmaceutically acceptable salt, e.g. mono-maleate or hydrochloride salt), which process comprises:
performing reaction step (i) using a reaction solvent comprising or consisting essentially of toluene and/or xylene (e.g. as described in the eleventh aspect of the invention); then
(ii) converting the resulting compound of formula (III) to the compound of formula (IV), which is 4-(tetrahydro-2H-pyran-4-yloxy)benzoic acid (e.g. as described herein either above or below); and then
either a) converting the 4-(tetrahydro-2H-pyran-4-yloxy)benzoic acid to a non-acid-chloride derivative thereof in which the carboxylic acid group has been activated, and then reacting this with 1-isopropyl-hexahydro-1H-1,4-diazepine;
or aa) converting the 4-(tetrahydro-2H-pyran-4-yloxy)benzoic acid to 4-(tetrahydro-2H-pyran-4-yloxy)benzoyl chloride and then reacting this with 1-isopropyl-hexahydro-1H-1,4-diazepine;
and optionally preparing a salt (e.g. pharmaceutically acceptable salt, e.g. hydrochloride salt or maleate (typically mono-maleate) salt) of 1-isopropyl-4-{[4-(tetrahydro-2H-pyran-4-yloxy)phenyl]carbonyl}hexahydro-1H-1,4-diazepine.

Process step (a) can e.g. be as described herein. Process step (aa) can e.g. be as described in the Background to the Invention (see Example 10 of WO 2005/040144 A1).

Step (ii) is a deprotection reaction. When P represents C₁₋₆ straight-chain alkyl (e.g. methyl, ethyl, n-propyl or n-butyl) or isopropyl or isobutyl (in particular methyl or ethyl), the reaction typically comprises treatment with a suitable alkali (e.g. aqueous), such as sodium hydroxide or potassium hydroxide (e.g. aqueous sodium hydroxide or potassium hydroxide solution), in a suitable solvent such as methanol (e.g. when P=Me), or ethanol (e.g. when P=Et), or toluene and/or xylene; e.g. at a suitable temperature, such as 70-100° C. (e.g. 95° C. or 80° C.) and/or at reflux, e.g. for 1 to 24 hours such as 2-6 hours or 2-3 hours; typically until the hydrolysis is substantially complete. In a particular embodiment, when the step (ii) reaction solvent is toluene and/or xylene, and the reaction comprises treatment with a suitable aqueous alkali such as aqueous sodium hydroxide or potassium hydroxide solution, the reaction comprises efficient (e.g. vigorous) stirring or mixing.

In a particular embodiment, a toluene and/or xylene solution containing the compound of formula (III), produced in step (i), is used directly in the subsequent hydrolysis step (ii), i.e. without isolation of the compound of formula (III), in particular when the subsequent step (ii) comprises alkaline (e.g. NaOH or KOH) hydrolysis of the ester. The reaction conditions for steps (i) and/or (ii) can in particular be as described herein, e.g. reaction step (i) can be carried out in the presence of triphenylphosphine and diisopropyl azodicarboxylate.

When P represents benzyl, the deprotection reaction (ii) can comprise hydrogenation.

According to a thirteenth aspect of the invention, there is provided a process for preparing a compound of formula (IV)

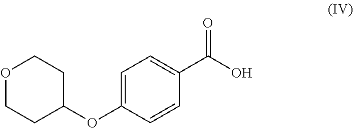

, which is 4-(tetrahydro-2H-pyran-4-yloxy)benzoic acid, wherein the process comprises:
(i) reacting the compound of formula (I), wherein P represents C₁₋₆ straight-chain alkyl (e.g. methyl, ethyl, n-propyl or n-butyl) or isopropyl or isobutyl (in particular methyl or ethyl), with 4-hydroxytetrahydropyran of formula (II) or a derivative thereof in which its OH group is activated, to prepare a compound of formula (III), wherein P has the same definition as in the compound of formula (I), and
(ii) hydrolysing the ester within the compound of formula (III), e.g. under alkaline conditions (e.g. using sodium hydroxide or potassium hydroxide, e.g. aqueous), to form the compound of formula (IV),
wherein the reaction steps (i) and (ii) are both carried out in a reaction solvent comprising or consisting essentially of toluene and/or xylene (in particular toluene).

“Xylene” can be o-xylene, m-xylene, p-xylene, or a mixture of xylenes.

In a particular embodiment of this thirteenth aspect of the invention, the toluene and/or xylene solution of the compound of formula (III) produced in step (i) is used directly in the subsequent hydrolysis step (ii), i.e. without isolation of the compound of formula (III), in particular when the subsequent step (ii) comprises alkaline (e.g. NaOH or KOH) hydrolysis of the ester. The reaction conditions for steps (i) and/or (ii) can in particular be as described herein, e.g. reaction step (i) can be carried out in the presence of triphenylphosphine and diisopropyl azodicarboxylate (e.g. ca. 1.3-2.0 e.g. ca. 1.5 mole equivalents of triphenylphosphine and/or diisopropyl azodicarboxylate with reference to compound (I); and/or e.g. the triphenylphosphine and the diisopropyl azodicarboxylate are present in substantially the same number of mole equivalents as each other).

In a fourteenth aspect of the present invention, there is provided a process for the preparation of 1-isopropyl-4-{[4-(tetrahydro-2H-pyran-4-yloxy)phenyl]carbonyl}hexahydro-1H-1,4-diazepine

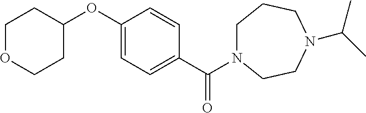

or a salt (e.g. pharmaceutically acceptable salt, e.g. mono-maleate or HCl salt) thereof, which process comprises:
performing reaction steps (i) and (ii), wherein the reaction steps (i) and (ii) are both carried out in a reaction solvent comprising or consisting essentially of toluene and/or xylene (e.g. as described hereinabove e.g. in the thirteenth and/or eleventh aspects); and then
either a) converting the 4-(tetrahydro-2H-pyran-4-yloxy)benzoic acid to a non-acid-chloride derivative thereof in which the carboxylic acid group has been activated, and then reacting this with 1-isopropyl-hexahydro-1H-1,4-diazepine;
or aa) converting the 4-(tetrahydro-2H-pyran-4-yloxy)benzoic acid to 4-(tetrahydro-2H-pyran-4-yloxy)benzoyl chloride and then reacting this with 1-isopropyl-hexahydro-1H-1,4-diazepine;
and optionally preparing a salt (e.g. pharmaceutically acceptable salt, e.g. hydrochloride salt or maleate (typically mono-maleate) salt) of 1-isopropyl-4-{[4-(tetrahydro-2H-pyran-4-yloxy)phenyl]carbonyl}hexahydro-1H-1,4-diazepine.

Process step (a) can e.g. be as described herein. Process step (aa) can e.g. be as described in the Background to the Invention (see Example 10 of WO 2005/040144 A1).

Compounds of formula (I) are either commercially available (for example, methyl 4-hydroxybenzoate is available from Aldrich), or they may be prepared from commercially available compounds using standard methodology. 4-Hydroxytetrahydropyran of formula (II) is commercially available, e.g. from Aldrich.

To prepare 1-isopropyl-hexahydro-1H-1,4-diazepine, as the dihydrochloride or bis-trifluoroacetate salt, from tert-butyl-hexahydro-1H-1,4-diazepine-1-carboxylate, see for example Intermediates 1A, 1B, 2 and 3 herein.

### Pharmaceutical Compositions, Doses, Dosage Regimens, and Uses

When used in therapy, the 1-isopropyl-4-{[4-(tetrahydro-2H-pyran-4-yloxy)phenyl]carbonyl}hexahydro-1H-1,4-diazepine or the pharmaceutically acceptable salt thereof (e.g. mono-maleate or hydrochloride salt) is usually formulated in a pharmaceutical composition. Such compositions can be prepared using various procedures.

The present invention further provides a pharmaceutical composition (e.g. adapted for oral administration) comprising 1-isopropyl-4-{[4-(tetrahydro-2H-pyran-4-yloxy)phenyl]carbonyl}hexahydro-1H-1,4-diazepine mono-maleate (e.g. a crystalline form thereof, such as crystalline Form 1 thereof) and a pharmaceutically acceptable carrier.

The present invention further provides a process for preparing a pharmaceutical composition, wherein the composition comprises 1-isopropyl-4-{[4-(tetrahydro-2H-pyran-4-yloxy)phenyl]carbonyl}hexahydro-1H-1,4-diazepine

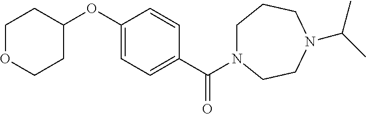

or a pharmaceutically acceptable salt thereof (e.g. mono-maleate salt such as crystalline Form 1 thereof, or hydrochloride salt) and a pharmaceutically acceptable carrier,
wherein the process comprises mixing the 1-isopropyl-4-{[4-(tetrahydro-2H-pyran-4-yloxy)phenyl]carbonyl}hexahydro-1H-1,4-diazepine or the pharmaceutically acceptable salt thereof with a pharmaceutically acceptable carrier (e.g. for example at ambient temperature and/or atmospheric pressure). This process can be in addition to (e.g. carried out after) the other processes of the invention.

The pharmaceutical composition is usually adapted for oral, parenteral or rectal administration and, as such, may be in the form of a tablet, a capsule, an oral liquid preparation, a powder, granules, a lozenge, a reconstitutable powder, an injectable or infusible solution or suspension, or a suppository. An orally administrable pharmaceutical composition, such as a tablet or capsule, is generally preferred.

The dose, e.g. oral dose, of the 1-isopropyl-4-{[4-(tetrahydro-2H-pyran-4-yloxy)phenyl]carbonyl}hexahydro-1H-1,4-diazepine or the pharmaceutically acceptable salt thereof, e.g. for use in the treatment or prophylaxis of any of the disorders mentioned in the Background and/or mentioned in the list of disorders/diseases below, in particular for use in the treatment or prophylaxis (e.g. treatment) of cognitive impairments in a disease such as Alzheimer's disease or a related neurodegenerative disorder or schizophrenia or attention deficit hyperactivity disorder, and/or comprised in a pharmaceutical composition, can for example vary in the usual way with the seriousness of the disorders, the weight of the sufferer, and/or other similar factors. However, as a general guide, in one embodiment a suitable unit dose (e.g. oral unit dose) of 0.01 to 1000 mg, for example 0.05 to 1000 mg or in particular 0.05 to 200 mg, of the 1-isopropyl-4-{[4-(tetrahydro-2H-pyran-4-yloxy)phenyl]carbonyl}hexahydro-1H-1,4-diazepine or the pharmaceutically acceptable salt thereof (measured as the “free base” compound), may be used, for example in a pharmaceutical composition (e.g. in an oral pharmaceutical composition, and/or e.g. in a unit dose form) of the invention. In one embodiment, such a unit dose is for administration once a day, e.g. orally and/or to a mammal such as a human; alternatively such a unit dose may be for administration more than once a day, for example two or three times a day, e.g. orally and/or to a mammal such as a human. Such therapy may extend for a number of weeks, months or years.

1-Isopropyl-4-{[4-(tetrahydro-2H-pyran-4-yloxy)phenyl]carbonyl}hexahydro-1H-1,4-diazepine hydrochloride was tested using substantially the histamine H3 functional antagonist assay disclosed on pages 47-49, and in particular page 48 line 26 to page 49 line 7, of WO 2009/030716 A1 (Glaxo Group Limited). The result is expressed as a functional pKᵢ (fpKᵢ) value. A functional pKᵢ is the negative logarithm of the antagonist equilibrium dissociation constant as determined in the H3 functional antagonist assay using membrane prepared from cultured H3 cells. The result given is an average of a number of experiments. A sample(s) of 1-isopropyl-4-{[4-(tetrahydro-2H-pyran-4-yloxy)phenyl]carbonyl}hexahydro-1H-1,4-diazepine hydrochloride exhibited H3 antagonism with a fpKᵢ of approximately 8.3 (as the mean of 12 experiments, which had a range of measured fpKᵢ values of from 7.7 to 9.3).

See also pages 50-52, pages 53-59 and FIGS. 10-14 of WO 2009/030716 A1 (Glaxo Group Limited) for the results of rat ex vivo binding studies (rat brain histamine H3 receptor occupancy) and pig-PET studies (positron emission tomography, showing pig brain H3 receptor occupancy profiles over time) on inter alia 1-isopropyl-4-{[4-(tetrahydro-2H-pyran-4-yloxy)phenyl]carbonyl}hexahydro-1H-1,4-diazepine hydrochloride.

The invention also provides 1-isopropyl-4-{[4-(tetrahydro-2H-pyran-4-yloxy)phenyl]carbonyl}hexahydro-1H-1,4-diazepine mono-maleate, in particular crystalline Form 1 thereof, for use (e.g. by oral administration) in the treatment or prophylaxis of:
a neurodegenerative disorder (in particular cognitive impairment(s) in a neurodegenerative disorder), wherein the neurodegenerative disorder can in particular be dementia (such as Alzheimer's disease, Lewy body dementia, vascular dementia or Parkinson's disease dementia), mild cognitive impairment, age-related memory dysfunction or cognitive ageing;
schizophrenia, in particular cognitive impairment(s) associated with (or in) schizophrenia; or
attention deficit hyperactivity disorder (ADHD), in particular cognitive impairment(s) therein;
epilepsy;
neuropathic pain;
fatigue and/or excessive daytime sleepiness, e.g. fatigue associated with multiple sclerosis;
depression (e.g. major depressive disorder); or
anxiety;
   in a mammal (e.g. human).

Preferably, the mono-maleate, in particular crystalline Form 1 thereof, is for use in the treatment or prophylaxis (e.g. treatment) of cognitive impairment(s); in particular:
cognitive impairment(s) in a neurodegenerative disorder [e.g. dementia (such as Alzheimer's disease, Lewy body dementia, vascular dementia or Parkinson's disease dementia), mild cognitive impairment, age-related memory dysfunction or cognitive ageing]; or
cognitive impairment(s) in (associated with) schizophrenia; or
cognitive impairment(s) in attention deficit hyperactivity disorder;
   in a mammal (e.g. human).

The invention also provides the use of 1-isopropyl-4-{[4-(tetrahydro-2H-pyran-4-yloxy)phenyl]carbonyl}hexahydro-1H-1,4-diazepine mono-maleate, in particular crystalline Form 1 thereof, in the manufacture of a medicament for use (e.g. by oral administration) in the treatment or prophylaxis of any of the above-mentioned diseases or disorders, in a mammal (e.g. human).

The invention also provides a method of treatment or prophylaxis (e.g. treatment) of any of the above-mentioned diseases or disorders in a mammal (e.g. human) in need of such treatment or prophylaxis, comprising administering (e.g. orally) to the mammal an effective amount of 1-isopropyl-4-{[4-(tetrahydro-2H-pyran-4-yloxy)phenyl]carbonyl}hexahydro-1H-1,4-diazepine mono-maleate, in particular crystalline Form 1 thereof.

Particular pharmaceutical compositions and/or oral dosage forms containing the active compound or salt (e.g. the mono-maleate salt of the invention), which can optionally be used, e.g. for oral dosage forms (e.g. tablets) containing 0.01 mg to 1 mg of the active compound or salt (measured as the free base), can be as follows.

In one particular embodiment of the invention, a dosage form for oral administration comprises a carrier tablet, wherein the carrier tablet is at least partially (e.g. partially) covered by a film comprising 1-isopropyl-4-{[4-(tetrahydro-2H-pyran-4-yloxy)phenyl]carbonyl}hexahydro-1H-1,4-diazepine

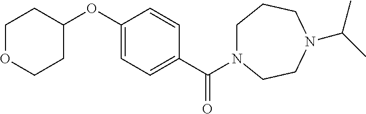

or a pharmaceutically acceptable salt thereof (e.g. the mono-maleate salt of the invention such as crystalline Form 1 thereof).

Optionally, the film, which at least partially covers the carrier tablet, comprises a stabiliser that reduces degradation of the 1-isopropyl-4-{[4-(tetrahydro-2H-pyran-4-yloxy)phenyl]carbonyl}hexahydro-1H-1,4-diazepine or the pharmaceutically acceptable salt thereof in the dosage form, when compared to a comparable dosage form lacking said stabiliser. The stabiliser can for example be citric acid, malic acid, ascorbic acid and its salts, sodium bicarbonate, butylated hydroxyanisole, butylated hydroxytoluene and/or a combination(s) thereof; in particular citric acid.

Optionally, and separately or additionally, the film, which at least partially covers the carrier tablet, comprises hydroxypropylcellulose (H PC).

In the context of this pharmaceutical composition, the term “carrier tablet” refers to a tablet that is substantially free of 1-isopropyl-4-{[4-(tetrahydro-2H-pyran-4-yloxy)phenyl]carbonyl}hexahydro-1H-1,4-diazepine or a pharmaceutically acceptable salt thereof. Usually, the carrier tablet does not contain any therapeutic agent. Preferably, the carrier tablet has one or more recesses (e.g. two recesses on both opposing sides of the tablet, i.e. a biconcave tablet), and more preferably the film containing the active compound or salt is present in the recess or recesses in the carrier tablet.

### COMPOSITION EXAMPLE 1

### Preparation of Round Tablets Containing 0.01 mg 1-isopropyl-4-{[4-(tetrahydro-2H-pyran-4-yloxy)phenyl]carbonyl}hexahydro-1H-1,4-diazepine or a Pharmaceutically Acceptable Salt Thereof (Measured as the Free Base)

The core components are passed through a nominal 30 mesh screen and then blended together in a suitable blender and compressed on a rotary tablet press to produce round biconcave tablets with a diameter of 7.9 mm and an approximately 0.8 mm deep trough on both sides of the tablet. Compression is followed by de-dusting and metal checking. The tablets are then transferred to a coating pan and coated to a target 4% (w/w) gain. The composition of the carrier tablets is given below (with a film coat on the carrier tablet):

| Component | Weight (mg) |
|---|---|
| Core | |
| Microcrystalline cellulose (Avicel PH-102 ™) | 162 |
| Pregelatinized Starch (Starch 1500) | 16.2 |
| Magnesium Stearate | 1.8 |
| Total | 180 |
| Film Coat | |
| Opadry White YS-1-7003 ™ | 7.2 |
| Purified Water | qs* |
| Total | 187.2 |

*Removed during processing

To form a film containing the active compound or salt onto the coated carrier tablet(s), the following process is carried out:

A carrier solution is prepared by dissolving 5 g hydroxypropylcellulose (Grade EF; HPC), and 3 g anhydrous citric acid in methanol, filtering through a 10 micron filter and then bringing the final volume to 100 ml with methanol. 1-Isopropyl-4-{[4-(tetrahydro-2H-pyran-4-yloxy)phenyl]carbonyl}hexahydro-1H-1,4-diazepine or a pharmaceutically acceptable salt thereof (e.g. mono-maleate salt thereof such as crystalline Form 1 thereof) is dissolved in the carrier solution with a sonicator (and by also using a magnetic stirrer) until a uniform solution is obtained with a final concentration of 12.5 mg/g (w/w) (measured as the free base). 4 mg carrier solution is dispensed onto each coated carrier tablet in an array of carrier tablets. The tablets are dried in a forced air oven at about 50° C. for 10-20 minutes.

The composition of the finished tablets is as follows:

| | Weight |
|---|---|
| Component | (mg) |
| 1-isopropyl-4-{[4-(tetrahydro-2H-pyran-4- | 0.01 |
| yloxy)phenyl]carbonyl}hexahydro-1H-1,4-diazepine or a | |
| pharmaceutically acceptable salt thereof (e.g. mono-maleate salt | |
| thereof such as crystalline Form 1 thereof) (measured as the free | |
| base) | |
| Carrier (5% hydroxypropylcellulose/3% citric acid in methanol*) | 0.4 |
| Carrier tablet | 187.2 |
| Total | 187.6 |

*methanol is removed during manufacturing process

These tablets containing the film with the active compound or salt can optionally be further coated.

### COMPOSITION EXAMPLES 2, 3 AND 4

### Preparation of round tablets containing 0.02 mg, 0.05 mg or 0.5 mg1-isopropyl-4-{[4-(tetrahydro-2H-pyran-4-yloxy)phenyl]carbonyl}hexahydro-1H-1,4-diazepine or a Pharmaceutically Acceptable Salt Thereof (Measured as the Free Base)

Tablets containing 0.02 mg, 0.05 mg or 0.5 mg of 1-isopropyl-4-{[4-(tetrahydro-2H-pyran-4-yloxy)phenyl]carbonyl}hexahydro-1H-1,4-diazepine or a pharmaceutically acceptable salt thereof (measured as the free base) (e.g. the mono-maleate salt thereof such as crystalline Form 1 thereof) can be prepared in the manner described in Composition Example 1 except that the concentration of 1-isopropyl-4-{[4-(tetrahydro-2H-pyran-4-yloxy)phenyl]carbonyl}hexahydro-1H-1,4-diazepine or a pharmaceutically acceptable salt thereof in the carrier solution is varied.

### EXPERIMENTAL SECTION

The following non-limiting Examples illustrate one or more of the preparation processes of the invention, and the Intermediates illustrate the preparation of intermediates which can be used in one or more of the preparation processes of the invention.

Abbreviations, some of which are used herein, include:
eq equivalents
HPLC high performance liquid chromatography
h hour(s)
min minute(s)
GC gas chromatography
LCMS or LC/MS liquid chromatography/mass spectrometry
NMR nuclear magnetic resonance
¹H NMR¹H nuclear magnetic resonance, in which br=broad, m=multiplet, s=singlet, d=doublet, t=triplet, td=triplet of doublets, etc, and 1H or 2H=integral shows one hydrogen or two hydrogens, etc.
TLC thin layer chromatography
room temperature (ambient temperature): this is usually in the range of about 17 to about 25° C., or a sub-range within this range, except as disclosed herein.

### Intermediate 1

### Method A

### 1-tert-Butyl-4-isopropyl-hexahydro-1H-1,4-diazepine-1-carboxylate

tert-Butyl-hexahydro-1H-1,4-diazepine-1-carboxylate (10.0 g) was dissolved in dichloromethane (200 ml). Acetone (7.33 ml) was added and the reaction was left to stir for 5 min. Sodium triacetoxyborohydride (21.0 g) was then added and the reaction was stirred at room temperature for 16 h. The reaction mixture was washed with saturated potassium carbonate solution (2×200 ml). The organic layer was dried (magnesium sulphate) and evaporated to give the title compound as a clear oil (11.0 g).

### Intermediate 1

### Method B

### 1-tert-Butyl-4-isopropyl-hexahydro-1H-1,4-diazepine-1-carboxylate

tert-Butyl-hexahydro-1H-1,4-diazepine-1-carboxylate (25.06 g) was dissolved in acetonitrile (250 ml). Anhydrous potassium carbonate (34.5 g) and 2-iodopropane (63 g, 37 ml) were added and the mixture was heated at reflux for 18 h. The cooled mixture was filtered and the solids were washed with acetonitrile. The combined filtrates were evaporated and the residual oil was dissolved in diethyl ether, washed with water, sodium thiosulphate solution and brine, dried (Na₂SO₄) and evaporated to give the title compound as a light brown oil (29.8 g).

### Intermediate 2

### 1-Isopropyl-hexahydro-1H-1,4-diazepine dihydrochloride

1-tert-Butyl-4-isopropyl-hexahydro-1H-1,4-diazepine-1-carboxylate (11.0 g, e.g. which can be prepared as described in Intermediate 1 Method A or B) was dissolved in methanol (200 ml) and 4N HCl in dioxan (100 ml) was added. The reaction was stirred at room temperature for 2 h and then evaporated to give the title compound as a white solid (9.6 g). ¹H NMR δ (CDCl₃): 11.35 (1H, s), 10.22 (1H, s), 9.72 (1H, s), 4.15-3.52 (9H, m), 2.83-2.40 (2H, m), 1.47 (6H, d, J=6.24 Hz).

### Intermediate 3

### 1-Isopropyl-hexahydro-1H-1,4-diazepine bis-trifluoroacetate salt

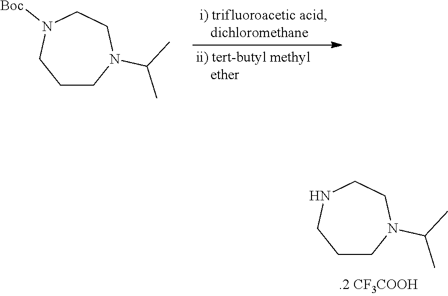

### Process Summary

All weights, volumes and equivalents are relative to 1-tert-butyl-4-isopropyl-hexahydro-1H-1,4-diazepine-1-carboxylate.

A solution of 1-tert-butyl-4-isopropyl-hexahydro-1H-1,4-diazepine-1-carboxylate (1 weight, e.g. which can optionally be prepared as described in Intermediate 1 Method A or B) in dichloromethane (14 volumes) is cooled to 0±3° C. Trifluoroacetic acid (5 weight, 3.4 volumes, 10 mole equivalents) is added over at least 30 min. The reaction mixture is then warmed to 30±3° C. and stirred at this temperature for at least 12 hours. Once the reaction is complete by TLC and GC, the mixture is evaporated to dryness on a rotary evaporator. Ethyl acetate (7 volumes) is added to the residue and the mixture is evaporated to dryness. This is repeated once. Ethyl acetate (0.5 volumes) is added to the resulting oil followed by tert-butyl methyl ether (10 volumes) and the mixture is stirred for 2 hours. The resulting solid is filtered off under vacuum and is washed with tert-butyl methyl ether (2×3 volumes). The solid is then dried in vacuo at 40° C. until a constant probe temperature is achieved, to give solid 1-isopropyl-hexahydro-1H-1,4-diazepine bis-trifluoroacetate salt.

### Example 1

### 4-(Tetrahydro-2H-pyran-4-yloxy)benzoic acid

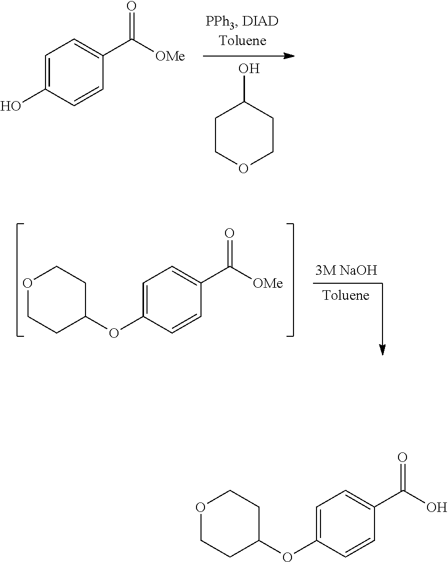

### Short Summary Process Description

All weights, volumes (“vol”) and equivalents are relative to methyl 4-hydroxybenzoate.

A solution of methyl 4-hydroxybenzoate (1 wt, 1 mole equivalent), triphenyl phosphine (2.6 wt, 1.5 mole equivalents), 4-hydroxytetrahydropyran (0.75 vol, 1.2 mole equivalents) in toluene (3.5 vol) under nitrogen is heated to 55° C. and diisopropyl azodicarboxylate (1.95 vol, 1.5 mole equivalents) is added dropwise over 60 minutes, maintaining the contents at 60±2° C. Following the addition, the reaction is stirred for 30 minutes, and then cooled to 0-5° C. The batch is then seeded with pre-prepared triphenylphosphine oxide-diisopropyl hydrazinedicarboxylate adduct, and then allowed to stir for a further 1 hour before filtering. The wet cake is washed with toluene (2×1 vol), and the combined mother liquors are transferred into a clean vessel. The toluene solution is washed with 2M sodium hydroxide solution (5 vol) at 0-5° C., and then 3M sodium hydroxide solution (5 vol) is added and the reaction is heated to 80° C. The reaction is stirred for at least 2.5 hours, until HPLC shows no starting material. The mixture is then cooled to 50° C. and toluene (5 vol) and water (5 vol) are added. The layers are allowed to separate, and the aqueous layer is washed with toluene (10 vol) and then acidified to pH1 with 2.5M HCl solution (7.5 vol). The resultant slurry is filtered and the wet cake is washed with water (2×2 vol). The title product is dried at about 50° C. in a vacuum oven with a nitrogen bleed to constant probe temperature.

### Detailed Process Description

1. Added methyl 4-hydroxybenzoate (1 wt, 482.3 g, available from Fluke) to Vessel 1.
2. Added 4-hydroxytetrahydropyran (0.75 vol, 362 mL, 1.2 mole equivalents, available from Sigma-Aldrich) to Vessel 1.
3. Added triphenyl phosphine (2.6 wt, 1253 g, 1.5 mole equivalents) to Vessel 1.
4. Purged Vessel 1 with Nitrogen.
5. Added toluene (3.5 vol, 1690 mL) to Vessel 1.
6. Heated contents to 55° C. with stirring.
7. Added diisopropyl azodicarboxylate (DIAD, 1.95 vol, 940 mL, 1.5 mole equivalents, available from Aldrich) to Vessel 1 via a peristaltic pump over 2 hours maintaining the contents temperature at 60±2° C.
8. Stirred contents of Vessel 1 at 60±2° C. for 50 min.
9. Sampled reaction mixture for HPLC analysis.
10. Cooled contents of Vessel 1 to 0-5° C.
11. Seeded batch with triphenylphosphine oxide-diisopropyl hydrazinedicarboxylate adduct (0.001 wt, 0.482 g)
12. Stirred contents of Vessel 1 for 81 min.
13. Filtered off biproduct over 5 min on a PTFE minifilter fitted with Whatman No. 113 wet strengthened filter paper (rough side up). Used 20 L Buchner flask as receiver.
14. Washed wet cake with toluene (2×ca. 1 vol, 2×490 mL) and sucked cake free of solvent.
15. Combined filtrate and cake washes were transferred to Vessel 2 via PTFE suck-up line.
16. Cooled Vessel 2 contents to 0-5° C.
17. Added 2M sodium hydroxide solution (5 vol, 2400 mL) to Vessel 2.
18. Stirred contents of Vessel 2 at 0-5° C. for 5 min before allowing the layers to settle.
19. Ran the lower aqueous layer into a labelled Schott bottle.
20. Added 3M sodium hydroxide solution (5 vol, 2410 mL) to Vessel 2.
21. Heated contents to 80° C., and stirred for 2 hours 45 min.
22. Monitored reaction by HPLC until hydrolysis is complete.
23. Cooled contents of Vessel 2 to 50° C., and then added toluene (5 vol, 2410 mL) to Vessel 2.
24. Added water (5 vol, 2410 mL) to Vessel 2.
25. Stirred contents at 50±5° C. for 5 min before allowing the layers to settle.
26. Ran the lower aqueous layer into a labelled Schott bottle for retention.
27. Ran the upper organic layer into a labelled Schott bottle for disposal.
28. Recharged aqueous layer from labelled Schott bottle to Vessel 2.
29. Added toluene (ca. 10 vol, 4900 mL) to Vessel 2.
30. Stirred contents at 50±5° C. for 5 min before allowing the layers to settle.
31. Ran the lower aqueous layer into a labelled Schott bottle for retention.
32. Ran the upper organic layer into a labelled Schott bottle for disposal.
33. Recharged aqueous layer to Vessel 2.
34. Added 2.5M aqueous hydrochloric acid (7.5 vol, 3620 mL) via peristaltic pump until pH1 is achieved.
35. Stirred the resulting slurry for 15 min.
36. Filtered off product on a PTFE mini filter fitted with Whatman 113 wet strengthened filter paper (rough side up). 10 min filtration time.
37. Washed filter cake with water (2×2 vol, 970 mL).
38. Dried the solid product in polythene lined steel trays covered with a muslin cloth, under vacuum and a nitrogen bleed, at 50° C. overnight and at 75° C. for a further 3 days.
39. Title product was obtained as an off-white solid (568.9 g).

### Analytical Data

¹H NMR (400 MHz, DMSO-d₆) delta ppm 1.55-1.64 (m, 2H) 1.95-2.03 (m, 2H) 3.49 (ddd, J=11.74, 9.41, 2.57 Hz, 2H) 3.85 (ddd, J=11.80, 4.34, 4.16 Hz, 2H) 4.69 (ddd, J=8.56, 4.65, 4.40 Hz, 1H) 7.03-7.09 (m, 2H) 7.84-7.90 (m, 2H), and 12.31 (br-s, 1H).

In an alternative to the above process, in step 37, the filter cake can be washed with toluene, instead of water, before the 50-75° C. vacuum drying of step 38.

### Example 2

### (Part A): 1-Isopropyl-4-{[4-(tetrahydro-2H-pyran-4-yloxy)phenyl]carbonyl}hexahydro-1H-1,4-diazepine

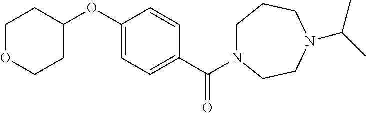

and

### (Parts B and C): 1-Isopropyl-4-{[4-(tetrahydro-2H-pyran-4-yloxy)phenyl]carbonyl}hexahydro-1H-1,4-diazepine hydrochloride

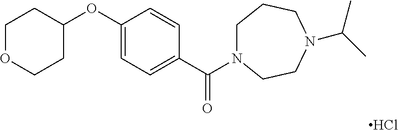

### Method

Carbonyl diimidazole (CD) (24 g, 0.8 weight, 1.1 equivalents) was dissolved in acetonitrile (about 300 ml, about 10 volumes) at a contents temperature of about 65° C. (jacket temperature 70° C.) with stirring under nitrogen in a three-necked flask equipped with a thermometer, condenser/nitrogen bubbler, and stopper. Dissolution was complete at about 35-40° C. 4-(Tetrahydro-2H-pyran-4-yloxy)benzoic acid (30 g, 1 weight, 1 equivalent; which can optionally be prepared as described in Example 1) was added portionwise via the side neck through a solid addition funnel, leading to vigorous gas evolution (CO₂). The addition was completed over 5-10 minutes. The addition funnel was rinsed with acetonitrile (about 45 ml, about 1.5 volumes) which was then added to the reaction mixture. The reaction mixture, activating the acid, was kept at 65° C. (jacket temperature 70° C.) for about 2 hours.

[Here, 1 volume=30 ml; volumes here are with respect to 30 g of input acid.]

Meanwhile, 1-isopropyl-hexahydro-1H-1,4-diazepine bis-trifluoroacetate salt (100 g, e.g. prepared as described in Intermediate 3) was dissolved in 2M aqueous sodium hydroxide solution (200 ml) and extracted with dichloromethane (2×200 ml). The organic extract was dried (Na₂SO₄) and evaporated to dryness to isolate 1-isopropyl-hexahydro-1H-1,4-diazepine as an oil (27.5 g, 0.916 weight, which if it were pure 1-isopropyl-hexahydro-1H-1,4-diazepine would be 1.43 equivalents).

All this oil was dissolved in acetonitrile (45 ml, 1.5 volumes) and the solution was transferred into the activated acid reaction mixture. The maximum total volume of acetonitrile in the reaction mixture at this stage is 400 ml.

The reaction mixture was conveniently left overnight to react at a contents temperature of 65° C. The reaction mixture was then allowed to cool; then it was clarified and split into three equal parts, Parts A, B and C, each part corresponding to 10 g of input 4-(tetrahydro-2H-pyran-4-yloxy)benzoic acid.

In each of these three Parts A, B and C, 1 volume=10 ml; volumes in these three Parts are with respect to 10 g of input acid (one third of the reaction).

### Part A: Preparation and isolation of 1-isopropyl-4-{[4-(tetrahydro-2H-pyran-4-yloxy)phenyl]carbonyl}hexahydro-1H-1,4-diazepine

The solvent was removed from Part A, the first part of the reaction mixture. Isopropyl acetate (10 volumes, 100 ml) was added and the mixture was washed with water (2×3 volumes, 2×30 ml), was dried over Na₂SO₄ and concentrated to 3 volumes (30 ml). Heptane (about 6 volumes, about 60 ml) was added which led to crystallisation of solid, and the mixture was stirred overnight. (That is, the free base was crystallised from about 1:2 isopropyl acetate:heptane.)

The slurry of free base from Part A was filtered and the separated solid was washed with heptane (3 volumes, 30 ml) and dried in a vacuum oven overnight to give 1-isopropyl-4-{[4-(tetrahydro-2H-pyran-4-yloxy)phenyl]carbonyl}hexahydro-1H-1,4-diazepine (the “free base”) (5.24 g).

This sample of free base was suspended in tert-butyl methyl ether (15 volumes with respect to the weight of free base to be recrystallised, about 75 ml) and the mixture was heated to 50° C. whereupon all of the free base dissolved to give a clear solution. Without clarifying it, the solution was allowed to cool gradually to room temperature to give a slurry. The slurry was filtered and the separated solid was dried in a vacuum oven overnight at 50° C. to give 1-isopropyl-4-{[4-(tetrahydro-2H-pyran-4-yloxy)phenyl]carbonyl}hexahydro-1H-1,4-diazepine as a solid (4.15 g).

This recrystallised sample of the free base appeared crystalline by XRPD (sharp peaks, data not shown), had a melting onset temperature of 93.6° C. (±1.5° C. for calorimeter error) by differential scanning calorimetry (DSC) (data not shown), and had a good gravimetric vapour sorption (GVS) profile (it adsorbed and/or absorbed approximately 0.4-0.5% w/w water (cf. minimum weight at ca. 30% RH starting humidity) across the 0-90% relative humidity range at 25° C. under nitrogen (data not shown)). Though the XRPD and GVS data appear good, the DSC melting onset temperature of 93.6° C. (±1.5° C. for calorimeter error, or ±5° C. including sample variation error) is relatively low as a melting onset temperature for an active pharmaceutical substance.

### Part B: Preparation and isolation of 1-isopropyl-4-{[4-(tetrahydro-2H-pyran-4-yloxy)phenyl]carbonyl}hexahydro-1H-1,4-diazepine hydrochloride

In parallel, Part B, the second part of the reaction mixture, was concentrated to 2.5 volumes (25 ml). Isopropanol (3 volumes, 30 ml) was added and the mixture was concentrated to 3 volumes (30 ml). Isopropanol (3 volumes, 30 ml) and water (2.0 ml, 0.2 volumes) were added and the resulting mixture (which contained ca. 3.2% water by volume) was heated to 65° C. A solution of HCl in isopropanol (5 to 6 N, 9 ml, 0.9 volumes) was added in one charge. About 10 minutes after the addition was complete, some crystals appeared. The mixture was cooled gradually to 20° C. over 4 hours. At 20° C. the slurry was so thick it would not stir. The slurry was further diluted with 3.2% water in isopropanol; a total of 60 ml (6 volumes) of (3.2% water in isopropanol) was added before a reasonably-stirrable slurry was obtained at 20° C.

The slurry was reheated to 60° C. to obtain a clear solution. This solution was cooled slowly; crystallisation commenced at about 38° C.; the mixture was cooled to 30° C.; and the slurry was stirred overnight at 30° C. The slurry was then cooled to room temperature slowly and was filtered. The resulting solid was washed with isopropanol (4 volumes, 40 ml) and dried in a vacuum oven overnight to give 1-isopropyl-4-{[4-(tetrahydro-2H-pyran-4-yloxy)phenyl]carbonyl}hexahydro-1H-1,4-diazepine hydrochloride as a solid (13.45 g). This can be named the “thermodynamic solid” hydrochloride salt (the result of slow cooling and crystallisation). HPLC suggests that this product contains one impurity present in an amount of ca. 3% (as measured by HPLC peak area). An XRPD analysis indicates that this “thermodynamic solid” hydrochloride salt is crystalline (XRPD has sharp peaks, data not shown).

### Part C: Preparation and isolation of 1-isopropyl-4-{[4-(tetrahydro-2H-pyran-4-yloxy)phenyl]carbonyl}hexahydro-1H-1,4-diazepine hydrochloride

In parallel, Part C, the third part of the reaction mixture, was concentrated to 2.5 volumes (25 ml). Isopropanol (3 volumes, 30 ml) was added and the mixture was re-concentrated to 3 volumes (30 ml). Isopropanol (7 volumes, 70 ml) and water (4 ml, 0.4 volumes) were added to give a mixture which contained ca. 3.8% water by volume. At room temperature, a solution of HCl in isopropanol (5 to 6 N, 9 ml, 0.9 volumes) was added in one charge, and shortly afterwards crystallisation commenced resulting in a thick slurry which was barely capable of being stirred. This slurry was filtered. The separated solid was washed with isopropanol (4 volumes, 40 ml) and was dried overnight in an oven at 50° C. and under vacuum to give 1-isopropyl-4-{[4-(tetrahydro-2H-pyran-4-yloxy)phenyl]carbonyl}hexahydro-1H-1,4-diazepine hydrochloride as a solid (12.4 g). This can be named the “kinetic solid” hydrochloride salt (the product produced by fast crystallisation). HPLC suggests that this product contains two impurities present in amounts of ca. 2% and ca. 13-14% respectively (as measured by HPLC peak area). XRPD data (not shown) appear to indicate that this “kinetic solid” hydrochloride salt from Part C comprises the same crystalline form as the “thermodynamic solid” hydrochloride salt from Part B.

To improve its purity, this “kinetic solid” hydrochloride salt was recrystallised from acetonitrile and water. The “kinetic solid” hydrochloride salt (11 g) was mixed with acetonitrile (90 ml) but did not completely dissolve in it even at 80° C. Water (1 ml) was added to the stirred slurry and slowly a clear solution was obtained. This stirred solution was allowed to cool to room temperature slowly and was stirred overnight. Filtration and drying gave 1-isopropyl-4-{[4-(tetrahydro-2H-pyran-4-yloxy)phenyl]carbonyl}hexahydro-1H-1,4-diazepine hydrochloride as a solid (9 g). No impurities were seen by HPLC.

### Example 3

### 1-Isopropyl-4-{[4-(tetrahydro-2H-pyran-4-yloxy)phenyl]carbonyl}hexahydro-1H-1,4-diazepine mono-maleate (crystalline Form 1)

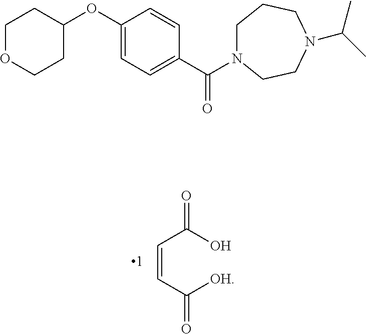

1-Isopropyl-4-{[4-(tetrahydro-2H-pyran-4-yloxy)phenyl]carbonyl}hexahydro-1H-1,4-diazepine (100 mg, prepared as described in Example 2 Part A, i.e. using the “free base” material which had been recrystallised from tert-butyl methyl ether) was fully dissolved in ethyl acetate (1 ml). Maleic acid (34 mg) was also dissolved in ethyl acetate (1 ml), and the two solutions were combined. On combination, a milky white precipitate was observed, but on stirring a solution was reformed. The solution was left to stand, and after about 1 hour a small amount of white solid had precipitated on the bottom of the vial. This was scratched into the solution, causing the precipitation of a significant quantity of white solid. The very thick slurry was diluted with further ethyl acetate (2 ml) and was subjected to a 0-40° C. temperature cycling program overnight.

After temperature cycling the white solid remained in the mixture. This solid was isolated from the mixture by filtration, and was dried over the weekend in vacuo at 40° C. 1-Isopropyl-4-{[4-(tetrahydro-2H-pyran-4-yloxy)phenyl]carbonyl}hexahydro-1H-1,4-diazepine mono-maleate was obtained as a solid (approx. 93 mg or approx. 95 mg).

A ¹H NMR (nuclear magnetic resonance) spectrum of this Example 3 product, as a solution in d6-DMSO, appeared to show an approximately stoichiometric quantity (1:1) of maleic acid present with respect to the molar amount of “free base”, and appeared to show no ethyl acetate present. The shift patterns of this solution ¹H NMR spectrum of the mono-maleate prepared in Example 3 were generally consistent with the shift patterns of the d6-DMSO solution ¹H NMR spectrum of the crystalline Form 1 mono-maleate prepared in Example 4 (see Example 5.5 for details).

### Example 3

### Solid-Form Infrared (IR) Data and Spectrum

A sample of the solid mono-maleate material prepared in Example 3 was subject to solid-form attenuated total reflectance infrared (IR) spectroscopy (see FIGS. 2 and 3), as described in more detail below.

A solid-form attenuated total reflectance (ATR) infrared (IR) spectrum of the crystalline Form 1 mono-maleate salt prepared in Example 3 was obtained on a Perkin Elmer Spectrum One FT-IR (Fourier Transform Infrared) spectrometer fitted with a Universal ATR Sampling Accessory (diamond/ZnSe). The sample was prepared by compressing the solid against the ATR cell. The spectrum was recorded with 4 scans at 4 cm⁻¹ resolution.

The solid-form attenuated total reflectance infrared spectrum of the crystalline Form 1 mono-maleate, which was obtained, is shown in FIG. 2 (the full IR spectrum, percent transmittance on vertical axis and wavenumber in cm⁻¹ on the horizontal axis) and in FIG. 3 (the “fingerprint region” of the IR spectrum, percent transmittance on vertical axis and wavenumber in cm⁻¹ on the horizontal axis), and comprises inter alia the following bands (peaks) at:

3021, 2958, 2949, 2932, 2864, 2847, 1700, 1622, 1604, 1575, 1509, 1464, 1422, 1393, 1375, 1353, 1341, 1308, 1297, 1280, 1247, 1234, 1205, 1178, 1169, 1153, 1132, 1115, 1089, 1069, 1048, 1017, 1005, 985, 962, 944, 908, 883, 869, 840, 828, 802, 784, 765, 725 and 685 cm⁻¹.

These ATR IR data were not collected on a regulated instrument, and the data were acquired for information use, since this batch was not to be used in a safety or clinical study. The Perkin Elmer Spectrum One FT-IR instrument was calibrated and serviced but not on a regular schedule or with full documentation. Based on the nature of the technique and instrumentation, it is considered that the data will show an accurate representation of the IR vibrational spectrum in terms of the bands observed, and their general profile and relative intensity. However, the band (peak) position may be less accurate, and at the time of filing it is considered that a likely error in each of the above-mentioned IR bands (peaks) is ±2 cm⁻¹.

In the ATR IR spectrum, relatively more intense IR bands (peaks) were observed at:

1700, 1622, 1464, 1422, 1353, 1247, 1234, 1089, 1048, 869, 840 and 765 cm⁻¹, with a variation for each peak of ±2 cm⁻¹.

### Example 4

### 1-Isopropyl-4-{[4-(tetrahydro-2H-pyran-4-yloxy)phenyl]carbonyl}hexahydro-1H-1,4-diazepine mono-maleate salt (crystalline Form 1)

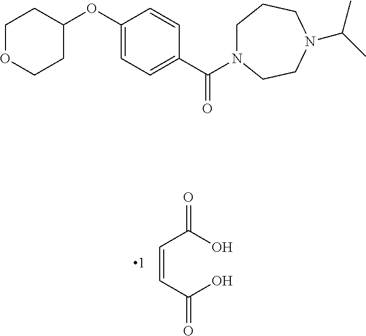

1-Isopropyl-4-{[4-(tetrahydro-2H-pyran-4-yloxy)phenyl]carbonyl}hexahydro-1H-1,4-diazepine (500 mg, 1.44 mmol; preferably prepared as described in Example 2 Part A that is using the free base material which had been recrystallised from tert-butyl methyl ether) was dissolved in ethyl acetate (5 ml) with stirring and sonication. Maleic acid (169 mg, 1.44 mmol, 1 mole equivalent) was also dissolved in ethyl acetate (5 ml) with sonication. The two solutions were combined dropwise. During the combination a white precipitate began to form, but immediately dissolved on stirring. The solution was then seeded with a few milligrams of a previously-synthesised batch of Form 1 crystalline 1-isopropyl-4-{[4-(tetrahydro-2H-pyran-4-yloxy)phenyl]carbonyl}hexahydro-1H-1,4-diazepine mono-maleate (prepared according to Example 3). The seed took, precipitation was seen, and over the next few minutes the slurry thickened. Further ethyl acetate (3 ml) was added, and the slurry was left to stir for about 60 minutes before being transferred to a 0-40° C. temperature cycling program and left for 3 nights (over the weekend).

After temperature cycling the thick slurry remained, and the resulting solid was isolated by filtration, and dried overnight in vacuo at 40° C. 1-Isopropyl-4-{[4-(tetrahydro-2H-pyran-4-yloxy)phenyl]carbonyl}hexahydro-1H-1,4-diazepine mono-maleate was obtained as a white solid (592 mg, 88% theoretical yield).

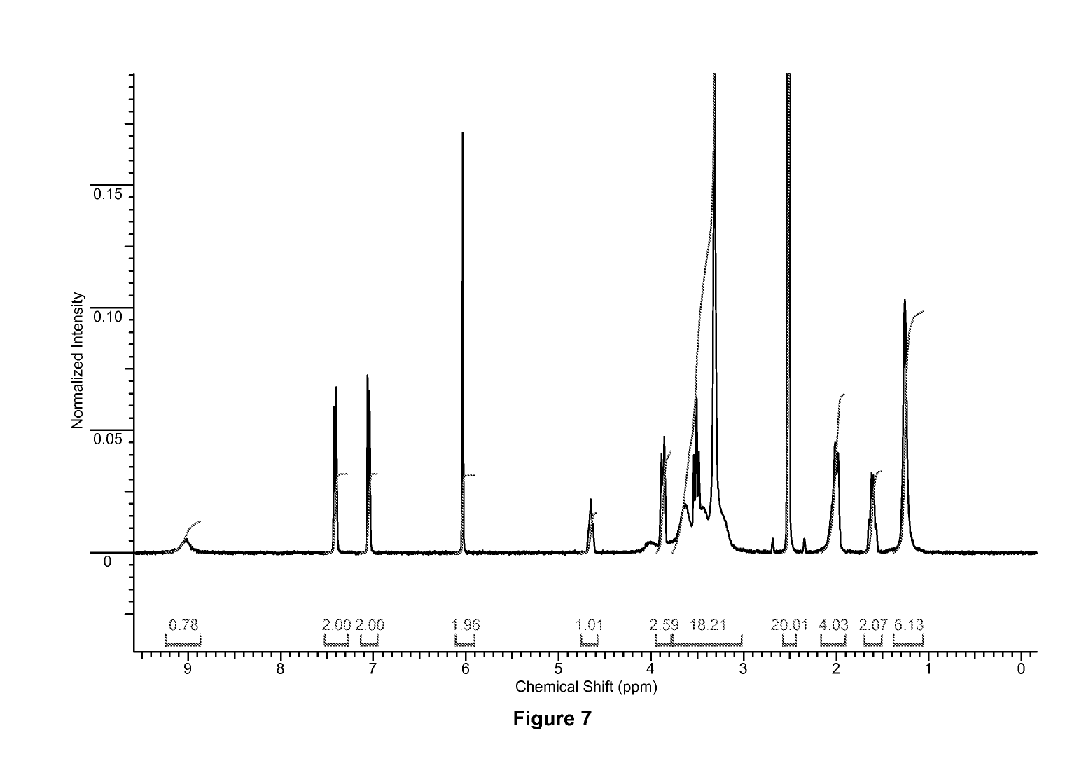

Samples of this material were removed for a range of analyses, including X-ray powder diffraction (XRPD) (see Table 1 below and FIG. 1), solid-form infrared (IR) spectroscopy (data not shown, but giving a solid-form IR spectrum consistent with that of the Form 1 crystalline mono-maleate produced in Example 3), differential scanning calorimetry (DSC) (see FIG. 4), thermo-gravimetric analysis (TGA), and gravimetric vapour sorption (GVS) (see FIG. 5). Also, a ¹H NMR (nuclear magnetic resonance) spectrum in d6-DMSO solution was run (see FIG. 7). See Example 5 below for analytical details.

Inter alia from the XRPD diffractogram shown in FIG. 1, which has sharp peaks, it appears that the solid mono-maleate salt is crystalline. This crystalline form of 1-isopropyl-4-{[4-(tetrahydro-2H-pyran-4-yloxy)phenyl]carbonyl}hexahydro-1H-1,4-diazepine mono-maleate is named as crystalline Form 1.

Gravimetric vapour sorption (GVS) analysis, which measures water sorption/desorption at different relative humidities, showed the sample of the Form 1 crystalline mono-maleate salt reversibly adsorbed and/or absorbed, and/or desorbed, approximately 0.4% w/w water across the 0-90% relative humidity range at 25° C. under nitrogen (see FIG. 5 and Example 5.3). This appears to represent an advantage, with respect to drug developability, over the solid hydrochloride salt of Example 2 Part C (the “kinetic” solid before purification by recrystallisation), which adsorbed and/or absorbed, and/or desorbed, higher quantities of water over the same relative humidity range (see FIG. 6 and Example 5.4 for comparative data).

### Example 5

### Spectroscopic and physical analyses of crystalline Form 1

### 1-isopropyl-4-{[4-(tetrahydro-2H-pyran-4-yloxy)phenyl]carbonyl}hexahydro-1H-1,4-diazepine mono-maleate prepared in Example 4

### Example 5.1

### X-ray powder Diffraction (XRPD) Data and Diffractogram for Crystalline Form 1 Mono-Maleate from Example 4 (FIG. 1)

X-ray powder diffraction (XRPD) data, on the crystalline Form 1 of 1-isopropyl-4-{[4-(tetrahydro-2H-pyran-4-yloxy)phenyl]carbonyl}hexahydro-1H-1,4-diazepine mono-maleate prepared in Example 4, were acquired on a Phillips PANalytical X'Pert Pro powder diffractometer model PW3040/60, serial number DY1850, equipped with an X′Celerator detector (available from PANalytical UK, 7310 IQ Cambridge, Waterbeach, Cambridge CB25 9AY, United Kingdom). The acquisition conditions were: radiation: Cu Kα (copper K-alpha), generator tension: 40 kV, generator current: 45 mA, start angle: 2.0° 2θ, end angle: 40.0° 2θ, step size: 0.0167° 2θ. The time per step was 31.750s. The sample was prepared by mounting a few milligrams of sample on a Si (silicon) wafer (zero background) plate, resulting in a thin layer of powder. The XRPD data were collected at room temperature and at atmospheric pressure in air.

Potentially characteristic peak positions, in degrees two-theta (2θ), and calculated d-spacings, in Angstroms (Å), are summarised in Table 1 below. These were calculated from the raw data using Highscore software (available from PANalytical UK, 7310 IQ Cambridge, Waterbeach, Cambridge CB25 9AY, United Kingdom). Experimental error in the peak positions is approximately ±0.1° two-theta (2θ). Relative peak intensities will vary due to preferred orientation.

FIG. 1 shows the XRPD diffractogram.

TABLE 1
XRPD data for crystalline Form 1 mono-maleate from Example 4

| | d-spacing/ |
|---|---|
| 2θ/° | Å |
| 9.2 | 9.6 |
| 11.8 | 7.5 |
| 13.4 | 6.6 |
| 14.7 | 6.0 |
| 15.8 | 5.6 |
| 16.2 | 5.5 |
| 17.0 | 5.2 |
| 18.5 | 4.8 |
| 19.8 | 4.5 |
| 20.3 | 4.4 |
| 20.5 | 4.3 |
| 21.0 | 4.2 |
| 21.3 | 4.2 |
| 27.1 | 3.3 |
| 27.4 | 3.3 |
| 27.8 | 3.2 |
| 28.4 | 3.1 |
| 30.2 | 3.0 |

As can be seen from FIG. 1, of the peaks listed in Table 1, seven XRPD peaks, which are of medium or strong intensity and/or which are believed most likely to be seen in a sample of test material containing the crystalline Form 1 mono-maleate, and some or all of which are more likely to be characteristic of the crystalline Form 1, are observed at the following degrees two-theta (2θ) values:

9.2±0.1°, 13.4±0.1°, 17.0±0.1°, 18.5±0.1°, 19.8±0.1°, 21.3±0.1°, and 27.8±0.1°.

### Example 5.2

### Differential Scanning Calorimetry (DSC) (FIG. 4), Thermo-Gravimetric Analysis (TGA), and Karl Fischer Water Content Analysis, for Crystalline Form 1 Mono-Maleate from Example 4

The differential scanning calorimetry (DSC) thermogram, of the crystalline Form 1 of 1-isopropyl-4-{[4-(tetrahydro-2H-pyran-4-yloxy)phenyl]carbonyl}hexahydro-1H-1,4-diazepine mono-maleate prepared in Example 4, was obtained using a TA Q1000 calorimeter, serial number 1000-0126 (available from TA Instruments Ltd, The Fleming Centre, Fleming Way, Manor Royal, Crawley RH10 9NB, United Kingdom), and the data processed using TA Universal Analysis software (available from TA Instruments Ltd, UK). The sample was weighed into an aluminium pan, a pan lid placed on top and lightly crimped without sealing the pan. The experiment was conducted at atmospheric pressure and under a flow of nitrogen using a heating rate of 10° C. min⁻¹.

The DSC thermogram obtained is illustrated in FIG. 4.

As shown in FIG. 4, the major endotherm, which is sharp, has an enthalpy of 104 J/g, and has an onset temperature of 149.2° C. with an estimated error of ±5° C. The onset temperature is 149.2±1.5° C. based on the calorimeter measurement error (±1.5° C.) for this sample, but a wider error of ±5° C. is specified to allow for variation between different crystalline Form 1 samples.

The onset temperature of a DSC endotherm is calculated as the temperature at which a linear extrapolation of the up-curve of the endotherm (i.e. of the lower-temperature portion of the endotherm, e.g. of the left-hand portion of the endotherm in FIG. 4) meets the baseline of the DSC thermogram.

Hence, the crystalline Form 1 mono-maleate is thought to have a melt onset temperature of 149.2±5° C. (or, for example, 149.2±1.5° C., for the tested sample).

In the DSC thermogram (FIG. 4) of the crystalline Form 1 mono-maleate salt prepared in Example 4, it can be seen that there is no endotherm due to loss of water, which would usually be seen if the crystalline form were a hydrate. Additionally, thermo-gravimetric analysis (TGA) of the crystalline Form 1 mono-maleate salt prepared in Example 4 (data not shown) does not show a significant weight loss before melting. These two data appear to suggest that the crystalline Form 1 mono-maleate salt prepared in Example 4 is an anhydrate rather than a hydrate.

A Karl Fischer analysis of water content was carried out on the crystalline Form 1 mono-maleate salt prepared in Example 4 (at normal United Kingdom room humidity). This Karl Fischer analysis reported a water content of less than 0.1% w/w.

Hence, it is thought, from the DSC, TGA and Karl Fischer data and also the GVS data shown herein, that crystalline Form 1 of 1-isopropyl-4-{[4-(tetrahydro-2H-pyran-4-yloxy)phenyl]carbonyl}hexahydro-1H-1,4-diazepine mono-maleate is not a hydrate. Instead, it is thought to be an anhydrate, that is a substantially anhydrous crystalline form.

### Example 5.3

### Gravimetric Vapour Sorption (GVS), for Crystalline Form 1 Mono-Maleate from Example 4 (FIG. 5)

Gravimetric vapour sorption (GVS) data were acquired on a Hiden IGA-Sorp analyser model (available from Hiden Isochema Ltd., 231 Europa Boulevard, Gemini Business Park, Warrington WA5 7TN, United Kingdom), generating a full sorption/desorption isotherm using water vapour perfusion at 25° C. under nitrogen gas and at varying relative humidities (RH).

23 milligrams of a sample of the crystalline Form 1 of 1-isopropyl-4-{[4-(tetrahydro-2H-pyran-4-yloxy)phenyl]carbonyl}hexahydro-1H-1,4-diazepine mono-maleate prepared in Example 4 were placed into a clean and dry tared sample mesh pan and weighed using the IGA-Sorp internal balance.

The GVS data at 25° C. for the crystalline Form 1 mono-maleate are illustrated in FIG. 5.

The target relative humidity (RH) ranges for the GVS data shown in FIG. 5 were:

(i) starting from about 30% RH and increasing to about 90% RH in increments of about 10% RH (results shown as the solid line in the middle and on the right of FIG. 5), and then
(ii) decreasing from about 90% RH to 0% RH in increments of about 10% RH (results shown as the dotted line in FIG. 5), and then
(iii) increasing from 0% RH to about 30% RH, in increments of about 5% RH up to ca. 10% RH, and then in increments of about 10% RH from ca. 10% to ca. 30% RH (results shown as the solid line on the left of FIG. 5).

The weight of the sample was recorded after each ca. 10% RH increment. The point of equilibrium, after each ca. 10% RH increment, was automatically determined using a 97% asymptote setting.

It can be seen from FIG. 5 that the sample of the crystalline Form 1 mono-maleate prepared in Example 4 reversibly adsorbs and/or absorbs, and/or desorbs, approximately 0.4% w/w (ca. 0.40-0.43% w/w) of water (relative to the minimum mass at ca. 0-5% RH) across the 0% to 90% RH range at 25° C. under nitrogen. This small weight change as RH varies is a good physical property for drug development, e.g. for storage, for quantification, for weighing, and/or for formulation e.g. into tablets, and/or for the storage, handling and/or physical properties of compositions such as tablets formed from the crystalline Form 1 mono-maleate.

### Example 5.4

### Comparative Data: Gravimetric Vapour Sorption (GVS) for Solid 1-isopropyl-4-{[4-(tetrahydro-2H-pyran-4-yloxy)phenyl]carbonyl}hexahydro-1H-1,4-diazepine hydrochloride salt from Example 2, Part C (FIG. 6)

Gravimetric Vapour Sorption (GVS) data, on the solid 1-isopropyl-4-{[4-(tetrahydro-2H-pyran-4-yloxy)phenyl]carbonyl}hexahydro-1H-1,4-diazepine hydrochloride from Example 2, Part C (the “kinetic” solid hydrochloride salt produced by fast crystallisation, and before purification by recrystallisation), were acquired on a DVS-1 SMS model (available from Surface Measurement System (SMS)), generating a full sorption/desorption isotherm using water vapour perfusion at 25° C. under nitrogen gas and at varying relative humidities (RH).

22 milligrams of the specified solid hydrochloride salt sample were placed into a clean and dry tared sample glass pan and weighed using the DVS-1 internal balance.

The GVS data at 25° C. for the specified solid hydrochloride salt sample are illustrated in FIG. 6.

The target relative humidity (RH) ranges for the GVS data shown in FIG. 6 were:

(i) starting from about 30% RH and increasing to about 90% RH in increments of about 10% RH (results shown as the line in the middle and on the right of FIG. 6, going from ca. 7-8% weight gain cf. minimum mass at ca. 30% RH, to ca. 14-15% weight gain cf. minimum mass at ca. 90% RH), and then
(ii) decreasing from about 90% RH to 0% RH in increments of about 10% RH (results shown as the line in FIG. 6, starting from ca. 14-15% weight gain cf. minimum mass at ca. 90% RH, going towards the left almost horizontally to ca. 14% weight gain at ca. 30% RH, and then going rapidly down to 0% weight gain at 0% RH), and then (iii) increasing from 0% RH to about 30% RH in increments of about 10% RH (results shown as the line on the bottom left of FIG. 6, going from 0% weight gain at 0% RH to about 2% weight gain at ca. 30% RH).

The weight of the sample was recorded after each ca. 10% RH increment. The point of equilibrium was automatically determined using a 0.02 dm/dt setting.

It can be seen from FIG. 6 that the sample of the specified solid hydrochloride salt (the “kinetic” solid hydrochloride salt produced by fast crystallisation, and before purification by recrystallisation) reversibly adsorbs and/or absorbs, and/or desorbs, approximately 14-15% w/w water (relative to the minimum mass at 0% RH) across the 0% to 90% RH range at 25° C. under nitrogen. When increasing to about 90% RH, starting from about 30% RH, the weight gain (relative to the minimum mass at 0% RH) increases from about 7-8% w/w at about 30% RH to about 14-15% w/w at about 90% RH, for this sample of the specified solid hydrochloride salt (see FIG. 6).

Hence, it is thought, from the GVS data shown in FIG. 6, that the specified solid hydrochloride salt of 1-isopropyl-4-{[4-(tetrahydro-2H-pyran-4-yloxy)phenyl]carbonyl}hexahydro-1H-1,4-diazepine (the “kinetic” solid hydrochloride salt produced by fast crystallisation, and before purification by recrystallisation), has a greater tendency to absorb/adsorb and/or desorb water as the relative humidity changes.

It is concluded, from FIG. 6, that this is likely to lead to the specified solid hydrochloride salt (the “kinetic” solid hydrochloride salt produced by fast crystallisation, and before purification by recrystallisation) being subject to greater weight changes as the relative humidity of the external environment changes, compared to the crystalline Form 1 mono-maleate salt whose GVS data shown in FIG. 5. This large weight gain/loss is likely to cause practical problem(s) for the physical properties, storage, measurement, quantification, and/or formulation of the specified solid hydrochloride salt (the “kinetic” solid hydrochloride salt, before purification by recrystallisation), and/or for the physical properties, storage, et al. of pharmaceutical compositions such as tablets formed from it.

FIG. 5 shows that this weight gain/weight loss problem with the specified solid hydrochloride salt (the “kinetic” solid hydrochloride salt, before purification by recrystallisation) is capable of being overcome by using the crystalline Form 1 of 1-isopropyl-4-{[4-(tetrahydro-2H-pyran-4-yloxy)phenyl]carbonyl}hexahydro-1H-1,4-diazepine mono-maleate as the active pharmaceutical substance instead of the specified solid hydrochloride salt thereof, and in pharmaceutical compositions such as tablets.

### Example 5.5

### ¹H NMR (Nuclear Magnetic Resonance) Solution Spectrum of Crystalline Form 1 Mono-Maleate from Example 4 (FIG. 7)

A ¹H NMR (nuclear magnetic resonance) spectrum of the crystalline Form 1 of 1-isopropyl-4-{[4-(tetrahydro-2H-pyran-4-yloxy)phenyl]carbonyl}hexahydro-1H-1,4-diazepine mono-maleate prepared in Example 4, was obtained as a solution in d6-DMSO (d6-dimethyl sulfoxide) using a 400 MHz ¹H NMR spectrometer, and is shown in FIG. 7, in which the horizontal axis shows the chemical shift in ppm (parts per million). The ¹H NMR spectrum, in which only 0 to 10 ppm is reported, has peaks at delta (ppm) as follows (integrals/numbers of hydrogens given in brackets are mainly taken from FIG. 7): 9.03 (0.78H, broad s), 7.41 (2.00H, d), 7.05 (2.00H, d), 6.04 (1.96H, s), 4.65 (1.01H, m), 4.01 (about 0.7H, broad m), 3.87 (about 2.6H, m), 3.1 to 3.8 (about 18.2H, m, includes triplet at 3.51 ppm and strong peak at 3.32 ppm), 2.52 (20.0H, s), 2.00 (4.03H, broad d), 1.61 (2.07H, m), 1.26 (6.13H, s or m). These reported peaks include the peak due to incompletely-deuterated DMSO.

The solution ¹H NMR spectrum of the Example 4 mono-maleate shown in FIG. 7 and reported above:

(i) appears to show some shifting of peaks of the active drug compound (cf. those of the “free base”) indicating that salt formation is likely to have occurred,
(ii) appears to show that the maleic acid had also been shifted (cf. free maleic acid itself),
(iii) appears to show that an approximately stoichiometric quantity (1:1) of maleic acid is present with respect to the molar amount of “free base”, and
(iv) appears to show no ethyl acetate present.

The shift patterns of this d6-DMSO solution ¹H NMR spectrum of the crystalline Form 1 mono-maleate prepared in Example 4 were generally consistent with the shift patterns of the d6-DMSO solution ¹H NMR spectrum of the crystalline Form 1 mono-maleate prepared in Example 3 (data not shown).

1-39. (canceled)

40. A salt which is 1-isopropyl-4-{[4-(tetrahydro-2H-pyran-4-yloxy)phenyl]carbonyl}hexahydro-1H-1,4-diazepine mono-maleate.

41. The salt according to claim 40 in a crystalline form.

42. A crystalline form of the salt according to claim 40 having an X-ray powder diffraction diffractogram comprising four or more of the following peaks at substantially the following degrees two-theta values:
9.2±0.1°, 13.4±0.1°, 17.0±0.1°, 18.5±0.1°, 19.8±0.1°, 21.3±0.1°, and 27.8±0.1°;
wherein the X-ray powder diffraction diffractogram is measured with a X-ray powder diffractometer using copper K-alpha X-radiation and a step size of 0.0167° two-theta or less.

43. A crystalline form of the salt according to claim 40 having an X-ray powder diffraction diffractogram substantially as shown in FIG. 1, wherein the X-ray powder diffraction diffractogram is measured with a X-ray powder diffractometer using copper K-alpha X-radiation and a step size of 0.0167° two-theta or less.

44. A crystalline form of the salt according to claim 40 having a solid-form attenuated total reflectance infrared spectrum comprising five or more of the following peaks:
1700, 1622, 1464, 1422, 1353, 1247, 1234, 1089, 1048, 869, 840 and 765 cm⁻¹;
with a variation allowed for each peak of ±2 cm⁻¹.

45. A crystalline form of the salt according to claim 40 having a solid-form attenuated total reflectance infrared spectrum substantially as shown in FIG. 2.

46. A crystalline form of the salt according to claim 40 having a solid-form attenuated total reflectance infrared spectrum substantially as shown in FIG. 3.

47. A pharmaceutical composition comprising the salt according to claim 40 and a pharmaceutically acceptable carrier.

48. A process for the preparation of crystalline Form 1 of the salt according to claim 40, comprising:
(a) mixing 1-isopropyl-4-{[4-(tetrahydro-2H-pyran-4-yloxy)phenyl]carbonyl}hexahydro-1H-1,4-diazepine, a solvent which is a C₂₋₄alkyl C₂₋₄alkanoate, and maleic acid, under conditions in which the maleic acid and the 1-isopropyl-4-{[4-(tetrahydro-2H-pyran-4-yloxy)phenyl]carbonyl}hexahydro-1H-1,4-diazepine are dissolved in the solvent,
(b) optionally, seeding the resulting mixture with crystalline Form 1 of the salt according to claim 40,
(c) allowing or causing the crystalline Form 1 of the salt according to claim 40 to crystallize from the mixture,
(d) separating the crystalline Form 1 of the salt according to claim 40 from the solvent, and
(e) optionally, drying the crystalline Form 1 of the salt according to claim 40.

49. The process as claimed in claim 48, wherein the solvent is ethyl acetate.
